(19) United States
(12) Patent Application Publication
(10) Pub. No.: US 2016/0289750 A1
(43) Pub. Date: Oct. 6, 2016

(54) LOCALISED RCA-BASED AMPLIFICATION METHOD USING A PADLOCK-PROBE

(71) Applicant: OLINK AB, Uppsala (SE)

(72) Inventors: Ulf LANDEGREN, Uppsala (SE); Lei CHEN, Yangzhou (CN)

(21) Appl. No.: 15/036,394

(22) PCT Filed: Nov. 14, 2014
(86) PCT No.: PCT/EP2014/074692

§ 371 Date: May 12, 2016

(30) Foreign Application Priority Data
Nov. 14, 2013 (GB) 1320145.4

(51) Int. Cl. C12Q 1/68

(52) U.S. Cl. CPC C12Q 1/6844; C12Q 1/6876

(57) ABSTRACT

The present invention provides a method for performing a localised RCA reaction comprising at least two rounds of RCA, wherein the product of a second RCA reaction is attached, and hence localised, to a product of a first RCA reaction, said method comprising: (a) providing a concatemeric first RCA product comprising repeated monomers; (b) directly or indirectly hybridising to monomers of said first RCA product a circularisable oligonucleotide comprising target-complementary 3′ and 5′ end regions such that the 3′ and 5′ ends of said oligonucleotide hybridise in juxtaposition for ligation directly or indirectly to each other, wherein the target is a sequence in a monomer of said first RCA product or an intermediate molecule hybridised thereto, and wherein the target-complementary end regions of said circularisable oligonucleotide are 6 to 16 nucleotides in length; (c) directly or indirectly ligating the ends of said circularisable oligonucleotide to circularise the oligonucleotide, thereby to provide a template for a second RCA reaction, wherein when said ends are indirectly ligated (i) either a gap oligonucleotide is provided which hybridises to the monomers of the first RCA product in between the 3′ and 5′ ends of the circularisable oligonucleotide such that it may be ligated to the respective ends, or the hybridised 3′ end of the circularisable oligonucleotide is extended by a polymerase such that the extended 3′ end may be ligated to the hybridised 5′ end, and (ii) the total length of the region of the second RCA template directly or indirectly hybridised to the monomers is no longer than 32 nucleotides in length; and (d) performing a second RCA reaction using said second RCA template of (c) and a primer for said second RCA, to form a second RCA product, wherein in said second RCA reaction the second RCA template remains attached to the first RCA product, and thereby the second RCA product is attached to the first RCA product.

The present invention lies generally in the field of nucleic acid amplification by rolling circle amplification (RCA), and particularly in the concept of generating a localised, more than linear, amplification based on RCA in at least two rounds of amplification. The product of the second (or further) RCA reaction is not physically derived from the first (or earlier) RCA product and the second or further round of RCA amplification provides a means for amplifying the signal obtainable from a rolling circle amplification reaction. The method of the invention involves generating a second RCA product by RCA of a second, separate, RCA template. The second RCA template is provided by a so-called padlock probe, a circularisable oligonucleotide which hybridises to the first RCA product in such a manner that the ends of the oligonucleotide may be ligated to one another, directly or indirectly, to form a circularised oligonucleotide which is topologically linked (catenated or “padlocked”) to the first RCA product. Contrary to expectation, it was found possible to achieve efficient RCA of such a padlock probe topologically linked to a first RCA product, namely RCA at a useful level. Since the second RCA template is physically attached to the product of a first RCA reaction, and the primer for the second RCA, which is extended to form the second RCA product, is and remains attached (hybridised) to the second RCA template, a localised amplification reaction may be obtained in which the product of the second RCA reaction is attached to the product of the first. The present invention provides such a method, and has particular utility in the amplification of signals from detection assays based on detecting RCA products.

Rolling circle replication (RCR) is a mechanism used in nature for the replication of circular DNA molecules such as plasmids or viruses The reaction has been adopted as the basis for a laboratory method for amplifying circular molecules and has been demonstrated to be useful in a variety of assays which use or generate a circular nucleic acid molecule as a reporter, wherein the circular molecule is amplified (replicated) by RCA and the replicated or amplified circular nucleic acid molecule is detected. In other methods, desired, or target molecules may be circularised and amplified by RCA. Accordingly, rolling circle replication (RCR) is now commonly referred to as rolling circle amplification (RCA), and these terms are used interchangeably herein.

RCA relates to the synthesis of nucleic acid molecules using a circular single stranded nucleic acid molecule, e.g. an oligonucleotide, as rolling circle template and a strand-displacing polymerase to extend a primer which is hybridised to the circular template(the strand displacing activity displaces the primer and effectively causes the circle to “roll”). The primer may in certain typical assays be provided by a target nucleic acid (RNA or DNA) molecule. The addition of a polymerase and nucleotides starts the synthesis reaction, i.e. polymerisation. As the rolling circle template is endless, the resultant product is a long single stranded nucleic acid molecule composed of tandem repeats that are complementary to the rolling circle template (i.e. a concatemer).

In practice, RCA reactions often utilise linear nucleic acid molecules, e.g. oligonucleotides such as padlock probes as described in more detail below, which are manipulated to generate circular nucleic acid template molecules, typically by ligating the ends of the nucleic acid molecule together, e.g. using a ligase enzyme. For instance, the ends of the nucleic acid molecule may be brought into proximity to each other by hybridisation to adjacent sequences on a target nucleic acid molecule which acts as a ligation template. The formation of the circular nucleic acid molecule allows it to be copied in a RCA reaction. This reaction may be initiated by adding a primer to the closed circle or a primer may be generated from the target nucleic acid molecule, i.e. ligation template. The initial primer therefore forms part of the RCA product. This can be particularly advantageous because it may allow localised detection of the target nucleic acid, i.e. in embodiments where the nucleic acid molecule used to prime RCA is immobilized, the RCA product will also be immobilized.

Thus, the RCA product may remain as a string of tandemly repeated complementary copies of the nucleic acid circle, a concatemer, which can be particularly useful for in situ detection, but may also be detected in homogenous (“in solution”) assays. For instance, a RCA reaction may result in a 1000-fold amplification of the circle in just 1 hour (based on a circle consisting of about 100 nucleotides). Thus, the RCA of a circular oligonucleotide may result in a RCA product that forms a bundle or “blob” of DNA that can be about 1 μm in diameter. The product, i.e. blob, may be detected by the hybridisation of nucleic acid probes conjugated to fluorescent labels which allows the blob to be visualised by fluorescence microscopy or flow cytometry. In other embodiments, the RCA products may be reduced to monomers by digestion with a restriction enzyme or a ribozyme, which are then detected.

Due to the ability of the RCA reaction to generate a readily detectable signal it is useful as a reporter system for detection of any nucleic acid molecule in a sample, which may be a target nucleic acid molecule (i.e. a nucleic acid molecule to be detected, or where the nucleic acid molecule is the “analyte” of the assay), or it may be a nucleic acid molecule which is to be detected as a marker (or proxy) for the presence of the target analyte. Thus the RCA reaction has been used to detect directly target nucleic acids in cell and tissue samples, e.g. in situ, i.e. localised, detection of nucleic acid molecules, which is of significant interest both for research and diagnostic purposes. However, RCA assays are not limited to use in heterogeneous formats and may also find utility in homogeneous assays (see e.g. WO 2009/037659).

RCA has also been utilised in methods for the detection of other analytes, i.e. analytes other than nucleic acid molecules such as proteins, peptides etc. In this respect, a variety of assays have been developed in which a nucleic acid molecule may be used to directly or indirectly tag or label a target analyte in a sample and detection of the nucleic acid molecule serves to indicate the presence of the analyte in the sample. In some methods a new nucleic acid molecule may be generated in a sample (i.e. a nucleic acid molecule that was not present in the original sample and was not one of the components added to the sample) when one or more molecules that interact with, e.g. bind to, the target analyte. The detection of the generated nucleic acid molecule is indicative of the analyte in a sample.

Various methods based upon detecting such a proxy or marker nucleic acid molecule using an RCA reaction as part of the detection strategy are well described in the art, including for example, immuno-RCA, assays using padlock probes and proximity probe assays which generate a circular nucleic acid molecule. In all these cases, the methods rely on providing or generating a circular nucleic acid molecule which may then be used as a substrate (template) for an RCA reaction, and the RCA product may then be detected as a substitute for detecting the target analyte directly.

Immuno-RCA involves labelling an antibody for a specific target analyte with a nucleic acid molecule. Typically, the target analyte is captured on a substrate, e.g. with a first antibody and contacted with the antibody/nucleic acid complex. A circular (or circularisable) oligonucleotide is hybridized to the nucleic acid molecule conjugated to the antibody (the oligonucleotide may be pre-hybridized or added after the antibody has been allowed to interact with the target analyte). Excess antibody may be washed away before the sample is subjected to an RCA to amplify the circular/circularised oligonucleotide. The nucleic acid molecule conjugated to the antibody is used as the primer for RCA. Thus, the RCA product is tethered to the antibody that is interacting with the target analyte, thereby allowing localised detection of the analyte in the sample, e.g. in a cell or tissue sample.

A proximity assay relies on the principle of “proximity probing”, wherein an analyte is detected by the binding of multiple (i.e. two or more, generally two or three) probes, which when brought into proximity by binding to the analyte (hence “proximity probes”) allow a signal to be generated. Typically, at least one of the proximity probes comprises a nucleic acid domain linked to the analyte-binding domain of the probe, and generation of the signal involves an interaction between the nucleic acid moieties and/or a further functional moiety which is carried by the other probe(s). Thus signal generation is dependent on an interaction between the probes (more particularly by the nucleic acid or other functional moieties/domains carried by them) and hence only occurs when the probes have bound to the analyte, thereby lending improved specificity to the detection system. The concept of proximity probing has been developed in recent years and many assays based on this principle are now well known in the art. For example, proximity ligation assays (PLAs) rely on proximal binding of proximity probes to an analyte to generate a signal from a ligation reaction involving or mediated by (e.g. between and/or templated by) the nucleic acid domains of the proximity probes.

The nucleic acid domains of the proximity probes when in proximity may template the ligation of one or more added oligonucleotides to each other, including an intramolecular ligation, to circularise one or more added linear oligonucleotides, to form a nucleic acid circle, based on the so-called padlock probe principle, as described for example by Landegren et al. in WO 99/49079. In such a method the ends of the added linear oligonucleotide(s) are brought into juxtaposition for ligation by hybridising to one or more circularisation templates provided by the nucleic acid domain of one or more proximity probes. Various such assay formats are described in WO 01/61037.

It will accordingly be evident that RCA may be of utility in the specific detection of any nucleic acid molecule in a sample, regardless of whether it is the “original” target analyte in a sample or it is a “proxy” target analyte generated by the interaction of specific detection molecules, e.g. proximity probes, with the target analyte, e.g. protein. RCA may also be useful in the detection of amplified nucleic acid molecules. For instance, in samples in which the target nucleic acid molecule is present in low amounts, e.g. rare transcripts, RCA can be used to “enhance” detection by increasing the amount of nucleic acid that is available to be detected.

Various modifications of the basic RCA reaction have been proposed, including to provide a more than linear amplification, for example to improve sensitivity in assays based upon detecting an RCA product.

Many of these methods include further amplification of the signal generated in the first RCA reaction, that is amplification based on the first RCA product. In general these are focused on techniques which use the product of the first RCA reaction as a template for primer extension. For example, in the hyperbranched RCA reaction (HBRCA), a primer is hybridised to each tandem repeat in the concatemeric RCA product generated by the first RCA reaction. The primers are then extended using the first RCA product as a template. As each primer is elongated it runs into and displaces the product of a downstream primer to generate a single-stranded tandem repeat of the sequence of the original first RCA reaction product. Further, the primer for the first RCA reaction may then hybridise to the tandem repeats in the generated (displaced) single strands and be extended to form further displacing strands, and so on. Thus, alternate strand copying and strand displacement processes generate a continuously expanding pattern of DNA branches, and by virtue of the strand displacement, also a discrete set of free DNA fragments comprising double-stranded pieces of the unit length of the circle, and multiples thereof. This is described by Lizardi in Nature Genetics 1998, 19, 225-2323 and in WO 97/19193.

A similar method is the DNA cascade reaction, as described by Koch in WO 97/20948, but in this case the primer extension time is controlled so that copying of the strands does not proceed to the end and the displaced strands remain attached to the template (RCA product).

In WO 03/012199 a method, termed the circle-to-circle (C2C) method, based on repeat RCA reactions, is described, which may also be used for amplifying the signal generated from a first RCA reaction. Again, the first generation RCA product is used as a template for extension of a primer hybridised to said product. However, in this method the first generation RCA product is cleaved into monomers (each monomer corresponding to one tandem repeat in the concatemeric product), which are circularised and then used as RCA templates in a further round of RCA. Since in this method the first RCA product is cleaved, there can be no localisation of the second RCA products to the first product.

Whilst methods for signal amplification based on carrying out secondary amplifications of primary RCA products have been described, there is a continuing need to develop assays with increased sensitivity and/or performance, e.g. with stronger signal, more rapid signal generation, and/or improved signal to noise ratio. In particular, there is a need to develop assays in which the secondary signal may be localised to the first, for example but not only in the case of in situ detection processes. It may in certain cases be useful or desirable to couple and co-localise signal detection based on the primary detection product, to signal detection based on a secondary detection product.

The present invention is directed to addressing this need, and is based on the concept of performing a second RCA reaction, which is dependent upon a first RCA reaction, but which does not amplify the first RCA product, in order to amplify the signal which may be generated by the first RCA reaction (in other words to generate more “signal product” by the second RCA, which is detected to generate the signal). An important feature of the present method is that the second RCA product is and remains physically attached to the first RCA product, in order that the signal from the second RCA product is localised to the first RCA product.

In U.S. Pat. No. 5,854,033 Lizardi describe a method termed nested ligation-mediated RCA (nested LM-RCA). LM-RCA involves generating an RCA product based on a principle analogous to padlock probing, in which a circularisable oligonucleotide (termed an “open circle probe”, but essentially a padlock probe) is hybridised to a target nucleic acid sequence (if present) and is then ligated to form a circle. After ligation a primer is hybridised to the circle and an RCA reaction is preformed to generate a linear RCA product comprising tandemly repeated complementary copies of the circle (termed the “tandem sequence DNA; TS-DNA). In the nested LM-RCA reaction, it is suggested that in order to amplify the signal, a further circularisable oligonucleotide may be hybridised to the TS-DNA, ligated, and subjected to a second round of RCA. However, actual performance of such a reaction is not demonstrated, and furthermore it is suggested that in such a procedure the circularised oligonucleotide would become displaced during the RCA reaction, and hence separated, from its target (to which it was hybridised). Accordingly, where a localised signal is desired, for example for in situ methods, U.S. Pat. No. 5,584 033 proposes an alternative method is in which the first circularisable oligonucleotide (OCP) is ligated but not amplified, and the second circularisable oligonucleotide is hybridised to the first, circularised, oligonucleotide (OCP). Thus, it has up to now not been believed possible that RCA of a padlock probe bound to a first RCA product (TS-DNA) would result in a localised secondary RCA reaction.

Furthermore, it has up to now been believed that the topological linkage of a circularised padlock probe on its target molecule would inhibit RCA, (see Baner et al., 1998 Nucleic Acids Research 26(22), 5073-5078). Such topological inhibition may be relieved by cutting the target molecule to create a target 3′ end near to the circularised and catenated padlock probe, which allows the RCA to proceed. Thus, it was not thought possible that a localised secondary RCA reaction based on a padlock probe bound to a first RCA reaction product could be carried out.

Based on this belief, the present inventors developed instead a localised secondary RCA method, termed super RCA (sRCA), in which a primer is hybridised to the first RCA product, and is used to amplify a second RCA template circle which is not itself bound or hybridised to the first reaction product (British patent application Nos. 1220503.5 and 1309327.3 filed on 14 Nov. 2012 and 23 May 2013 respectively, corresponding to WO 2014/076209 A1).

However, contrary to expectations, by appropriate reaction design it was found that it was in fact possible to develop a localised secondary RCA-based amplification method using padlock probes. In particular, it was surprisingly found that by reducing the size of the target-hybridising portions (region) of the padlock probe (i.e. the target complementary end regions of the probe, namely the 3′ and 5′ end sequences), it was possible to perform RCA reactions on padlock probes bound and catenated (i.e. “padlocked”) on a first RCA product. Furthermore, as will be described in more detail in the Examples below, the results obtainable show the amount of and/or rate of amplification product production is such as to indicate that multiple padlocks bound along the length of a first RCA product are being replicated (subjected to RCA). Accordingly, it is believed that by carrying out a secondary RCA reaction using a padlock having target binding sites (target complementary regions) of a particular size range, secondary RCA of multiple padlocks bound along the length of a first RCA product may be obtained, and not just of one padlock bound at the free end of the first RCA product.

As noted above, a second RCA product is generated by RCA of the circularised padlock, which acts as the template for the second RCA reaction. A RCA primer may be provided pre-hybridised to the padlock probe, or it may be added after the padlock probe has been hybridised to the first RCA product, or after circularisation of the padlock probe (ligation). The second RCA product is produced by extension of the RCA primer, which, by virtue of its hybridisation to the template for the second RCA, is indirectly hybridised, and hence attached, to the first RCA product. The first RCA product is a concatemer comprising tandem repeat complementary copies of the template circle for the first RCA reaction (the “first RCA circle”), and the padlock probe which provides the RCA template for the second RCA is designed to bind to repeated copies of its cognate probe-binding sequence, repeated throughout the first RCA product. In other words, the first RCA product will comprise repeated binding sites for the padlock probe, at least one in each of the tandem repeats (“monomers” of the concatemer). Each such padlock when circularised can template a second RCA reaction, leading to increased, more than linear, amplification.

The method of the present invention allows the second RCA to be localised to the first RCA. As will be explained further below, this may have benefits in localised (spatial) detection of target analytes (e.g. in in situ detection procedures).

The strong signal amplification afforded by the second RCA reaction may increase the sensitivity of the method and provide the ability easily to detect or visualise the RCA products, for example at low microscopic magnification or with digital image scanning methods, or possibly even without the aid of microscopy. The strong signal from the increased amount of second RCA product may also open up the possibility of using other detection modalities, e.g. flow cytometry, in RCA-based detection assays, and thereby increase the instrument base possible to use in such assays. Signal amplification may be increased even further by a possible third or further generations of RCA all linked by virtue of the templates for each RCA generation, which, by binding to the RCA product of the preceding RCA reaction, link the previous RCA product to the next RCA product.

Further, since the second RCA reaction may be initiated during the first RCA reaction (i.e. as the first RCA product is being formed, or whilst the first RCA reaction is ongoing), a more rapid signal generation may be achieved, leading to faster assays.

In view of this coupling of two more RCA generations to yield both increased and faster signal amplification and the use of a padlock probe to provide the RCA template for successive RCA reactions, we have termed this new method padlock super RCA (padlock sRCA).

Accordingly in a first aspect, the present invention provides a method for performing a localised RCA reaction comprising at least two rounds of RCA, wherein the product of a second RCA reaction is attached, and hence localised, to a product of a first RCA reaction, said method comprising:
(a) providing a concatemeric first RCA product comprising repeated monomers;
(b) directly or indirectly hybridising to monomers of said first RCA product a circularisable oligonucleotide comprising target-complementary 3′ and 5′ end regions such that the 3′ and 5′ ends of said oligonucleotide hybridise in juxtaposition for ligation directly or indirectly to each other, wherein the target is a sequence in a monomer of said first RCA product or an intermediate molecule hybridised thereto, and wherein the target-complementary end regions of said circularisable oligonucleotide are 6 to 16 nucleotides in length;
(c) directly or indirectly ligating the ends of said circularisable oligonucleotide to circularise the oligonucleotide, thereby to provide a template for a second RCA reaction, wherein when said ends are indirectly ligated (i) either a gap oligonucleotide is provided which hybridises to the monomers of the first RCA product in between the 3′ and 5′ ends of the circularisable oligonucleotide such that it may be ligated to the respective ends, or the hybridised 3′ end of the circularisable oligonucleotide is extended by a polymerase such that the extended 3′ end may be ligated to the hybridised 5′ end, and (ii) the total length of the region of the second RCA template directly or indirectly hybridised to the monomers is no longer than 32 nucleotides in length.
(d) performing a second RCA reaction using said second RCA template of (c) and a primer for said second RCA, to form a second RCA product, wherein in said second RCA reaction the second RCA template (circularised oligonucleotide) remains attached to the first RCA product, and thereby the second RCA product is attached to the first RCA product.

Thus, in step (d) the second RCA product (extended second RCA primer) is hybridised to the second RCA template and hence attached to the first RCA product via, or by, the second RCA template. In other words, since it is hybridised to the second RCA template, the second RCA product is indirectly attached to the first RCA product. Accordingly, the fact that the second RCA template is not displaced from the first RCA product during the second RCA reaction serves to attach the second RCA product to the first RCA product.

Steps (a), (b), (c) and (d) may be performed simultaneously or sequentially, but preferably they are performed sequentially.

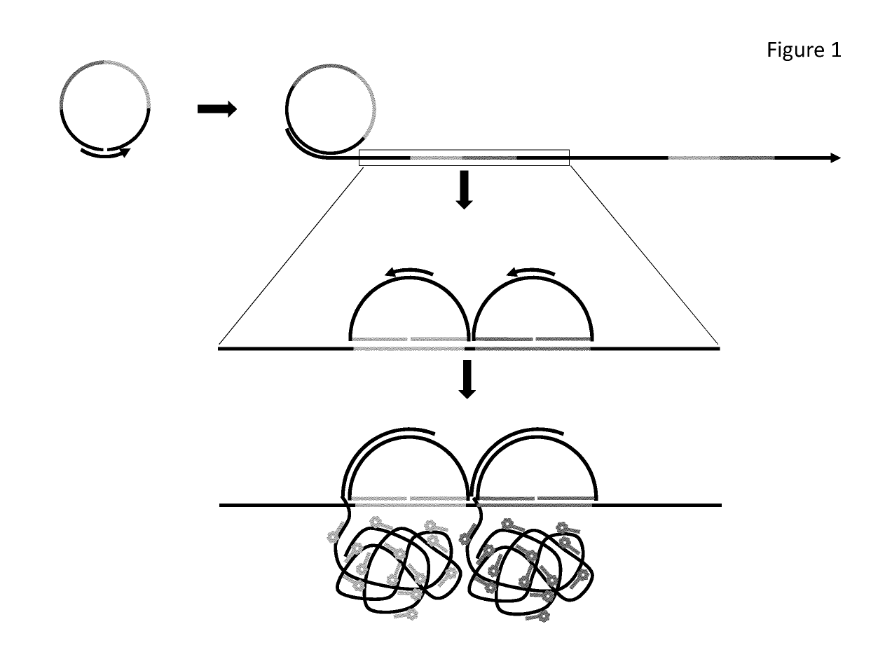

It will be recognised from the above definition of the invention that the circularisable oligonucleotide is in effect a padlock probe. That is, the circularisable oligonucleotide is a linear single-stranded nucleic acid molecule, as depicted for example in FIG. 1, which comprises target-complementary regions at its 3′ and 5′ ends. In particular the circularisable oligonucleotide does not have secondary structure, and more particularly does not comprise intramolecular double-stranded regions or stem-loop structures. The respective 5′ and 3′ ends of the circularisable oligonucleotide are ligated together, directly or indirectly, using the first RCA product, or an intermediate molecule hybridised thereto, as ligation template, thereby directly to form a circular template for the second RCA reaction (i.e the second RCA template is formed directly by the ligation reaction). More particularly, there is no intervening step of cleavage of the oligonucleotide to form the second RCA template.

When the oligonucleotide (probe) is circularised by ligation it becomes topologically linked, or catenated, to the molecule to which it has bound (hybridised), namely to the first RCA product or intermediate nucleic acid molecule hybridised thereto. Whilst in the past it has been believed that RCA of a circularised padlock probe may result in displacement of the probe, we have observed that in the method of the invention the padlocks do not become displaced and remain attached to their target (e.g. first RCA product). Whilst not wishing to be bound by theory, it is believed that binding of the padlock probes in tandem to multiple binding sites present in the target molecule, e.g. at least one in each of the tandemly repeated monomers of the concatemeric first RCA product (or an intermediate molecule bound thereto) prevents or limits diffusion of the circularised and bound (catenated) padlock along the nucleic acid strand. Furthermore the presence of multiple padlocks bound along the target molecule will block (i.e. prevent or inhibit any unwanted extension on the first RCA product as template. Each bound padlock probe will in effect act as displacement block, preventing or limiting any unwanted extension from displacing a downstream padlock probe. Such unwanted extension might for example occur from an unligated padlock probe, or other nucleic acid molecule present in the reaction mixture which hybridises non-specifically to the first RCA product.

The fact that the second RCA template (circularised oligonucleotide) remains (directly or indirectly) attached to the first RCA product is important in order to preserve the localisation of the second RCA product to the first RCA product. As noted above it is accordingly a feature of the method of the invention that no extension using the first RCA product as template can take place, or that any extension which does occur is limited such that there is no displacement of any downstream circularised oligonucleotides. More particularly, it is a further feature of the invention that any extension of any unligated circularisable oligonucleotides, or any unligated gap oligonucleotides if present, using the first RCA product as template is limited to avoid displacement of downstream circularised oligonucleotides. As explained above, a ligated and catenated padlock probe acts a block preventing any further extension, and displacement of any downstream probes. A catenated padlock is in effect an extension and/or displacement block.

Extension might also take place from non-specifically hybridised molecules, or from hybridised molecules degraded by exonuclease action, so as to leave a hybridised 3′ end capable of extension. As will be described in more detail below, in one embodiment such extension may be prevented or limited by incorporating an “exonuclease block” into nucleic acid reagents used in the method, to prevent exonuclease digestion from creating a primer capable of priming on the first RCA template, and/or by ensuring that no exonuclease activity is present in the reaction mixture, e.g. by using a polymerase enzyme with no exonuclease activity. Furthermore, as also will be described in more detail below, blocking groups and/or blocking oligonucleotides may be used, which are themselves blocked from extension and displacement, hybridised in between the padlock probes. In this way, any first RCA product-templated extension which does take place may be limited from extending into, and displacing any downstream-hybridised probes. However, the use of such exonuclease blocks or added displacement blocks is not essential, and in other embodiments they are not used.

By “circularisable” it is meant that the oligonucleotide which provides the second RCA template is in the form of a linear molecule having ligatable ends which may circularised by ligating the ends together directly or indirectly, i.e. to each other, or to the respective ends of an intervening (“gap”) oligonucleotide or to an extended 3′ end of the circularisable oligonucleotide. The second RCA template may thus be provided in two or more parts, namely two or more molecules (e.g. circularisable and gap oligonucleotides) which may be ligated together to form a circle.

The circularisable oligonucleotide is circularised prior to RCA by ligation, which is templated by the molecule to which the circularisable oligonucleotide has hybridised, i.e. the first RCA product or an intermediate molecule which has hybridised thereto. The circularisable oligonucleotide will comprise at its respective 3′ and 5′ ends regions of complementarity to corresponding cognate complementary regions (or binding sites) in the target molecule (namely the first RCA product or intermediate molecule hybridised thereto, which acts as the ligation template), which may be adjacent where the ends are directly ligated to each other, or non-adjacent, with an intervening “gap” sequence, where indirect ligation is to take place.

The first RCA product may be the product of a primary (i.e. initial) RCA reaction, or it may be the product of a further or later RCA reaction. The second RCA reaction may be a secondary or further, or later, RCA reaction. It will thus be understood that the method of the invention may involve multiple, successive rounds of RCA, e.g. two, three, four or more, wherein in each round a circularisable oligonucleotide (padlock probe) is used which is hybridised, directly or indirectly, to the reaction product of a previous round of RCA. Expressed in other words, the method of the invention may comprise repeating steps (a), (b), (c) and (d) one or more times.

It is a feature of the present invention that the first RCA product remains intact in the method, i.e. it is not cleaved (e.g. it is not cleaved into monomers). Furthermore the first RCA product in step (a) is single-stranded. More particularly in step (a) the first RCA product is provided in single-stranded form. Thus, “openers” or other molecules used to open up a portion of a double-stranded nucleic acid to allow a padlock-type probe to bind are not required and are not used according to the present invention.

Since an RCA product contains multiple repeat (or tandem) copies (or monomers) of a sequence (i.e. it is a concatemer of monomers), multiple probes (circularisable oligonucleotides) will be bound. Each monomer comprises at least one binding site for a circularisable oligonucleotide. Thus, the method of the invention more particularly comprises hybridising (directly or indirectly) a multiplicity of probes to the first RCA reaction product. As used herein the term “multiple” or “multiplicity” means two or more, e.g. at least 2, 3, 4, 5, 6, 10, 20, 30, 50, 70 or 100 or more. The probe is hybridised, directly or indirectly, to a binding site present in each repeat unit or monomer of the first RCA product (each repeat unit or monomer being a complementary copy of the circular RCA template used to produce the first RCA product (the “first RCA template”)). Thus, it will be seen that the circularisable oligonucleotide may comprise a sequence, e.g. a binding site or target complementary region which is identical to a sequence present in the first RCA template. It will be understood that whilst each of the repeat units or monomers of the product of the first RCA reaction comprise the binding site for the circularisable oligonucleotide (padlock probe), in practice not all of these binding sites may (or will) be occupied by a probe after probe hybridisation. It suffices that a number, or multiplicity, of such binding sites are bound by a probe. Thus, in the method of the invention the probe may hybridise to a probe-binding site present in a monomer of the first RCA product or to an intermediate nucleic acid molecule hybridised to a complementary sequence present in a monomer of the first RCA product. More specifically, the probe hybridises to a probe-binding site in at least one monomer of the RCA product or to an intermediate nucleic acid molecule hybridised to a complementary sequence present in at least one monomer of the first RCA product, but preferably in at least 2, 3, 4, 5, 6, 8, 10, 12, 15, 20, 50, 80 or 100 or more monomers or intermediate molecules. A monomer may comprise binding sites (cognate target-complementary regions) for more than one different padlock probe (circularisable oligonucleotide). Thus, each monomer may comprise multiple binding sites for the same or different probes.

The probe (circularisable oligonucleotide) may be hybridised directly to the first RCA product i.e. it binds directly to a probe-binding site present in the first RCA product which is complementary or “cognate” to a binding site in the probe. Alternatively, the probe may be hybridised indirectly, for example by binding to an intermediate nucleic acid molecule which is itself hybridised (directly or indirectly) to the first RCA product. In such an embodiment, the probe is bound to a complementary (or cognate) binding site present in the intermediate nucleic acid molecule. The intermediate nucleic acid molecule may be bound to the first RCA product by hybridising to a complementary (or cognate) binding site present in each repeat unit (or monomer) of the first RCA product). However, as indicated above, it may be the case that not every intermediate molecule, or not every probe-binding site in the intermediate molecule, will be bound by a probe.

In the method of the invention a primer is required for the second RCA reaction. It may be provided pre-hybridised to the circularisable oligonucleotide or it may be separately provided, for example after hybridisation and/or ligation of the oligonucleotide. A binding site for the second RCA primer may be provided in a region of the circularisable oligonucleotide which is different or separate from the target-complementary regions. A circularisable oligonucleotide/padlock probe may be viewed as having two target-complementary end regions (or “arms”) joined by an intervening region or domain which may be viewed as the “backbone” domain or region. A binding site for the second RCA primer may be provided in the backbone region.

A schematic illustrating a representative embodiment of the invention is presented in FIG. 1. The template for the first RCA is generated by circularising a first padlock probe by ligation, when it has hybridised to the primer for the first RCA. The first RCA reaction is then initiated to generate the first RCA product. After a certain time of rolling by the polymerase in the first RCA, the padlock probe for the second RCA is ligated on the first RCA product, and after this ligation to form the second RCA template, the circularised oligonucleotide starts to roll in the presence of the second RCA primer, which is hybridised to the backbone part of the second padlock, to generate the second RCA product. As depicted, there can be several second padlock probes ligated on the same monomer (repeat) of the first RCA product, depending on the size of the repeat and the padlock hybridisation length. After the second RCA, the second RCA product may be detected by labelling with one or more different detection probes labelled with different (distinguishable labels, e.g. different colours or fluorescent labels etc.

The method of the invention may be homogenous or heterogeneous. That is, it may be performed in solution, without a solid phase or support (i.e. without immobilisation of any reaction components) or it may be performed in an immobilised or solid phase-based format, particularly where the first RCA product is immobilised. Immobilisation of the first RCA product may be achieved in various ways. For example in an in situ assay the first RCA product may be formed in a first RCA reaction primed using a target (analyte) nucleic acid as first RCA primer. Here the first RCA product is attached to the target tissue sample which is itself fixed to a solid support. This may occur for example where a target nucleic acid is detected using a padlock probe. Alternatively, the first RCA may be primed by an nucleic acid domain of an immuno RCA or a proximity probe, which is bound to an immobilised (or fixed) analyte target. In other embodiments, the primer for the first RCA reaction may simply be immobilised to a solid support. Use of a heterogeneous, immobilised format allows washes to be readily performed, and hence for example allows for ready removal of unbound and/or unligated probes, and/or or other unreacted reaction components added, or spurious unwanted reactions, not physically attached to the surface. Thus, a heterogeneous, or solid phase-based method may readily be performed sequentially.

Since the present invention requires binding of the circularisable oligonucleotide directly or indirectly to a first RCA product and ligation thereon to form the second RCA template, the second RCA reaction is unable to take place unless the circularisable oligonucleotide has hybridised to the first RCA product i.e. unless the first RCA product is present. Thus, the second RCA reaction is dependent upon the presence of the first RCA product. In this way the specificity of the method may be improved. The second RCA reaction may thus be viewed as a target-dependent RCA reaction, wherein the target of the second RCA reaction is the first RCA reaction product.

Washing steps may also be used to improve specificity, for example by including one or more washing steps after probe hybridisation, and/or after the ligation step. This may readily be achieved in a solid phase or immobilised format by washing away any unbound and/or unligated reactants. Accordingly, in the absence of the first RCA product, the circularisable oligonucleotide has no “binding target” (i.e. no first RCA product or no intermediate nucleic acid molecule), and hence is not immobilised by binding to its target, and may be washed away. In this way the template is not available for the second RCA if the first RCA product is absent.

Accordingly, the method may be conducted such that unbound probe (that is probe (circularisable oligonucleotide) which has not hybridised directly or indirectly to the first RCA product) is removed before carrying out the RCA reaction of step (d). This may readily be achieved for example by carrying out the method in a solid phase format and washing after the step of probe addition and hybridisation. Alternatively or additionally washing, e.g. stringent washing, may be carried out after ligation step (c) to remove any unligated probes.

Alternatively, unbound and/or unligated circularisable oligonucleotides (probes) may be removed from the sample by enzymatic degradation in order to improve specificity after the ligation step. This may be performed by any means which preferentially degrades single-stranded linear oligonucleotides. In the absence of the first RCA product the circularisable oligonucleotide will not be hybridised to the RCA product (i.e. the 3′ and 5′ ends of the oligonucleotide will not be in juxtaposition for ligation, and hence the oligonucleotide will not be circularised. In this way unbound (and thus uncircularised) probes will be degraded, thus the template is not available for the second RCA if the first RCA product is absent. Furthermore, in the presence of first RCA product, background may be reduced by removing any remaining unhybridised probes. Thus an enzymatic degradation of unhybridised probes may be performed as a cleaning step. In this way, any subsequent amplification of unhybridised probes can be avoided, including both amplification of unligated probes and amplification by RCA of probes which may later become non-specifically ligated (i.e. non-target ligated probes). The present method may be performed in such a way that the unbound probe (circularisable oligonucleotide) which has not hybridised to the first RCA product (and thus not been circularised) is removed prior to carrying out the RCA reaction of step (d). This may be achieved by adding a component after step (c) which preferentially degrades linear oligonucleotides but does not degrade circularised oligonucleotide molecules. In a preferred embodiment, degradation may be performed by a component having 3′ exonuclease activity, preferably by a DNA polymerase enzyme having 3′ exonuclease activity, although a separately added exonuclease enzyme (e.g. a 3′ exonuclease enzyme) may alternatively or additionally be used. In a particularly preferred embodiment, the enzyme may be Phi29 polymerase. This may be performed by adding the component having 3′ exonuclease activity after ligation step (c) and prior to the addition of the primer required to initiate the second RCA. Alternatively or additionally, this step may be performed in the absence of dNTPs. Thus a component having 3′ exonuclease activity may be provided after the ligation of the circularisable oligonucleotide.

Alternatively, a component having exonuclease activity may be added after step (b) and before step (c), i.e. prior to the ligation step, to degrade any remaining unhybridised probes. Where a component having 3′ exonuclease activity is added before the ligation step, an exonuclease having strict single-stranded specific activity may preferably be used.

Whilst not wishing to be bound by theory, it is thought that the enhanced specificity and sensitivity associated with the use of a 3′ exonucleolysis step to remove unbound and/or unligated probe molecules may arise through the removal of unligated probes (circularisable oliogonucleotides), which may be attached to the first RCA product by at least their 3′ end, or which may be free in the sample. Unligated probes bound to the first RCA product would be capable of acting as a primer for extension using the first RCA product as a template. Spurious priming of the complement of the first RCA product formed in this way by free unligated probes or other DNA molecules in the sample may result in the ‘hyperbranched’ RCA amplification method mentioned in U.S. Pat. No. 6,183,960, and any amplification products generated in this way may be detectable by the detection probes used to detect the second RCA product, leading to an increase in the level of background signal generated. It is also anticipated that unligated probes may hybridise to the second RCA product and reduce the effectiveness of binding of the detection probes to the second RCA product, leading to a reduction in the level of signal generated. Thus the use of the 3′ exonucleolysis step may improve the signal:noise ratio of the method of the present invention.

It is contemplated that such enzymatic degradation may be performed in both homogeneous and heterogeneous reaction formats, however such a method may advantageously allow unbound (and thus uncircularised) probes to be removed before carrying out the RCA reaction of step (d) in a homogeneous (i.e. solution-phase) format in which the target analyte is not immobilised.

It is also understood that it may be desirable to inactivate the component having polymerase activity used to generate the first RCA product prior to the addition of the circularisable oligonucleotides to prevent the unwanted extension of unbound circularisable oligonucleotides (probes) in order to enhance the specificity of the present methods. If the polymerase is active during the ligation step, the uncircularised oligonucleotide may be degraded by 3′ exonuclease activity of the polymerase, or it may be extended by the polymerase, before it is ligated. In certain embodiments, this may be achieved by heat-inactivation prior to the addition of the circularisable oligonucleotides.

Thus in one embodiment, the method of the present invention may be carried out as follows:
i) Hybridisation and ligation of the first padlock probe to target DNA
ii) Rolling Circle Amplification of first padlock probe
iii) Inactivation of the polymerase
iv) Hybridisation and ligation of second padlock probe (circularisable oligonucleotide) on first RCA product
v) Removal of unligated second padlock probe by means of exonuclease
vi) Second RCA using a primer complementary to the second padlock probe, whilst the padlock remains attached to the first RCA product

It is also anticipated that in a further embodiment, the amount of the circularisable oligonucleotide (probe) used in the methods of the present invention may be titrated in such a way as to match the amount of the first RCA product formed. In this way, excess unbound probes may be avoided. However, it will be apparent that in such an embodiment it may also be desirable to remove excess unbound and/or unligated probes to further enhance the sensitivity of the detection assay.

Accordingly, the method is designed and may be conducted in such a way that production of the second RCA product is prevented or minimised in the absence of the first RCA product. More particularly, in the method of the invention, in the absence of the first RCA product production of the second RCA product is prevented or reduced to stochastic levels.

It will be understood that whilst the method is designed or conducted such that the second RCA reaction should not take place unless the first RCA product is present, it is the nature of such methods that non-specific reactions can occur, and absolute prevention cannot be guaranteed. Thus, certain undesired non-specific or random reactions or interactions may take place in the sample or reaction mixture, and these may lead to the generation of a second RCA product, for example primed by a component or nucleic acid molecule other than the probe/primer. This is what is meant by production of second RCA product at stochastic levels.

As noted above, a feature of the method of the invention is that the circularisable oligonucleotide (probe) is unable to prime extension using the first RCA product as template, or that any such extension which does take place is limited to avoid displacement of any downstream probes hybridised to the first RCA product. It will be understood of course that this requirement applies to any method component or reactant which is hybridised to the first RCA product. This will include any intermediate oligonucleotide molecules which are used.

To achieve this feature, various means and procedures may be used, singly or in combination, depending on the precise nature of the method. For example, in certain embodiments modifications (e.g. blocking groups or modified residues) can be incorporated into the probe (circularisable oligonucleotide) which inhibit polymerase and/or exonuclease action (i.e. which inhibit extension and/or degradation), or which inhibit strand displacement. Blocking oligonucleotides (e.g. with modifications as above) may be used to protect a 3′ end hybridised to the first RCA template (or intermediate molecule) from extension. For example, such blocking oligonucleotides may be hybridised to the first RCA template in between the probes of the invention. To prevent unwanted exonuclease digestion of any hybridised probes from creating a primer/template construct capable of priming on the first RCA product, in certain embodiments the presence of any reagents having exonucleolytic activity can be avoided, for example an exonuclease-deficient polymerase can be used. Washing steps may be used. For example, as noted above, any probes which have hybridised but not ligated may be removed by stringent washing (according to principles well known in the art). This is particularly applicable in the case of heterogeneous, or solid phase-based methods. Any combination of such means may be employed.

As noted above, in other embodiments exonuclease activity can be used. The addition of a component having 3′ exonuclease activity in conjunction with a DNA polymerase or DNA polymerase activity can prevent the RCA of any circularisable oligonucleotides (probes) which have become circularised (ligated) non-specifically (e.g. in the absence of the first RCA product or as a non-specific background reaction wherein ligation is templated by another molecule). In such embodiments which utilise a component having 3′ exonuclease activity, it is desirable that circularisable oligonucleotides (probes) do not comprise an exonuclease block.

A so-called exonuclease block, as indicated above, may be used (namely a modification which acts to block, or inhibit, exonuclease activity, particularly 3′ exonucleolytic activity). For example the probe may comprise such a block in the region between the 3′ end of the probe the 5′ hybridised region, e.g. a stretch of nuclease-resistant nucleotides.

Alternatively or additionally, the probe may be modified at or near a hybridised ligatable 5′ end to include a displacement block. In such a case any extension which does take place from the 3 ‘end will not be to displace the hybridised 5 ‘end (of the probe itself or of a downstream hybridised probe).

Modifications or blocking groups for inhibiting or blocking exonuclease and/or polymerase action (i.e. for inhibiting or blocking degradation and/or extension), as well as inhibiting or blocking displacement are well known in the art and described in the literature, and any of these may be used. Accordingly a nucleotide may be modified to include or incorporate a blocking group which may inhibit an enzyme (e.g. polymerase) from functioning, e.g. from binding. For example an exonuclease block may comprise a region of nuclease-resistant nucleotide residues. These may for example be 2′O-Me-RNA residues, Locked Nucleic Acid (LNA) residues, Peptide Nucleic Acid (PNA) residues, phosphothiate-modified nucleic acids, or a polyethylene-linker backbone moiety incorporated in between nucleotide residues. Abasic (apurinic or apyramidinic, or AP) sites may also be included as exonuclease blocks. Further the probe or probe component may in include a hairpin structure as an exonuclease block. There are several means of modifying nucleic acids so that they are exonuclease resistant and/or do not function as a primer and it is not intended that the methods of the invention are limited to the examples listed above.

Similarly, an extension or polymerase block may include any of the modified residues or blocks indicated above as exonuclease blocks. Any group or modification which inhibits binding of polymerase may be incorporated into the relevant region of the probe. For example, any groups having an intercalating function may be used, e.g. acridine groups.

A blocking oligonucleotide may include or incorporate any of the exonuclease or extension/polymerase blocks mentioned above. Blocking oligonucleotides which may inhibit unwanted extension reaction are described in the literature, for example in Olasagasti et al., 2010, Nature Nanotechnology, 5, 798-806 and in the Senior Thesis of Rashid, at the University of California, Santa Cruz, entitled Blocking Oligomer Design (3/10-3/11).

Displacement blocks are also known in the art and any reported or known displacement block may be used. A displacement block may comprise any stretch or region of modified nucleotide residues that form more stable hybrids with DNA and/or RNA than unmodified DNA and/or RNA. Such modifications include LNA, PNA, and 2’-0-Me RNA residues. Thus a stretch or region of such residues of sufficient length, or strength, to avoid displacement may be used. Abasic (AP) sites may be also be used. DNA clamps as reported in the literature may also be used.

Accordingly, in one possible embodiment, the method of the invention comprises the use of a circularisable oligonucleotide (probe) which incorporates (or comprises) one or more modified regions which act to inhibit degradation, extension and/or strand displacement, and/or blocking oligonucleotides are used (which may themselves incorporate such modified regions).

More particularly, in this embodiment, the modified region, as indicated above, acts to inhibit degradation, extension and/or strand displacement. Accordingly, the modified region may comprise or constitute an exonuclease block, an extension block and/or a displacement block. The modified region may comprise modified nucleotides and/or blocking groups and/or hairpin structures. Modified nucleotides include abasic nucleotides. Blocking oligonucleotides comprising one or more such modified regions may be added to hybridise to the first RCA product in such a manner that the probe is not able to extend using the first RCA product as template, or any such extension is limited to avoid displacement of any downstream probe hybridised to the first RCA product. For example, blocking oligonucleotides may be hybridised to the first RCA product in between a multiplicity of probes, e.g. such that there is at least one blocking oligonucleotide in between any two probes.

In other embodiments the use of such separate blocks is not necessary, and the ligated and catenated probes (circularised oligonucleotides) can themselves act as blocks to avoid any unwanted extension or displacement.

As noted above, one of the advantages for the present invention is that the second RCA may be initiated whilst the first RCA in ongoing, or in other words, as soon as the first RCA product has started to form. This leads to the advantage of faster signal generation. Thus signal amplification might optimally proceed as v²/2, compared to where v is the rate of nucleotide incorporation by the polymerase, compared to v for a single RCA, and hence at a multiple of the rate at which new RCA products are generated. This can speed up any RCA-dependent protocol, and may be of particular value for rapid detection assays. Further increases of signal strength or speed or both are possible with a further round of RCA initiating off and linked to the second RCA reaction product and so forth (e.g. a third, fourth, fifth . . . generation of RCA).

Thus, in one embodiment, the method of the invention may include as step (a) the step of generating a first RCA product, (or performing a first RCA reaction to produce a first RCA product). Steps (b), (c) and (d) may take place as soon as the first RCA product starts to form. Accordingly, steps (a) and/or (b) and/or (c) and/or (d) may be performed simultaneously or substantially simultaneously. In particular, step (b) may be performed simultaneously with step (a). Accordingly, in certain embodiments the reagents for the second RCA reaction, e.g. the probe and any additional reactants required may be added directly to the reagents for the first RCA reaction.

In other embodiments the steps of the method may be performed sequentially.

In another embodiment, as noted above, a further ‘cleaning’ step may be performed between the first and second RCA reactions. As mentioned above it is contemplated that this may comprise the addition of a component having 3′ exonuclease activity to the sample prior to the commencement of the second RCA reaction to remove any unbound and/or uncircularised circularisable oligonucleotides (probes). In such an embodiment, it is anticipated that the primer required for the initiation of the second RCA is not added before or contemporaneously with the component having 3′ exonuclease activity. Thus, after the first RCA step, and after hybridisation and ligation of the circularisable oligonucleotide, a polymerase having 3′ exonuclease activity (e.g. phi 29 polymerase may be added to allow degradation of unligated circularisable oligonucleotides. After this cleaning step, the primer for the second RCA reaction may be added to initiate the second RCA. Alternatively or additionally the exonuclease digestion step may be performed in the absence of dNTPs which are then added to initiate the second RCA. Accordingly, certain reagents for the second RCA reaction may be added directly to the reagents for the first RCA reaction, and certain reagents may be added subsequently. Alternatively, the cleaning step may be performed after hybridisation step (b) but before ligation step (c). The ligation step (c) may then be performed.

In further embodiments, the cleaning step may be performed using a separate exonuclease enzyme, which may be added after step (b) and/or after step (c). This may involve inactivating the exonuclease after the cleaning step, e.g. by heating. As noted above, the polymerase used for the first RCA reaction may be inactivated prior to the ligation step (c), and such various steps may be used in combination in different embodiments of the method. Polymerase, or polymerase and primer, for the second RCA reaction may for example be added after such inactivation step(s).

Although conditions suitable for performing the cleaning step (3′ exonucleolysis) will be known in the art, in a representative embodiment this may be performed using phi29 polymerase at 37° C. for around 20 minutes. Suitable times may vary however, and it is contemplated that this step may be performed for between 5-60 minutes, for instance 5, 6, 7, 8, 9, 10, or 15 minutes, or 25, 30, 35, 40, 45, 50 or 55 minutes.

The method of the invention relies upon multiple probes being able to hybridise to the first RCA product. Accordingly, it will be understood that the first RCA product needs to be available for probe hybridisation. This requirement is a feature of all RCA-based detection methods, where an RCA product is detected by hybridising a probe, e.g. a detection probe, to the product, and is well understood in the art. Thus, it may be advantageous for the first RCA product to have low secondary structure. However, this feature may be compensated for by performing the method in conditions which favour hybridisation, according to principles well known in the art. Thus, for example, the method can be performed in the presence of formamide e.g. in buffers containing formamide.

The first RCA product may be derived from the RCA of any nucleic acid (e.g. DNA or RNA) circle, or indeed the circle may be of any modified nucleic acid, as long as it is capable of templating a RCA reaction. The circle (first RCA template) may for example be a reporter DNA circle, namely from any RCA-based detection assay which uses or generates a nucleic acid circle (circular nucleic acid molecule) as a reporter for the assay. Thus the first RCA product may be the product of an immunoRCA or a proximity probe assay in which a circular nucleic acid molecule is generated, for example as discussed above, or it may be obtained by RCA of a circularised padlock probe. Alternatively, the first RCA template used to generate the first RCA product may be a circularised target (analyte) nucleic acid molecule. Circularisation of target nucleic acid molecules using circularisation adaptors (so-called “Selectors”) is described by us in WO 99/049079, WO 2003/012119 and WO 2005/070630.

As noted above, the length of the target-complementary regions of the padlock probe (circularisable oligonucleotide), or more particularly the length of the region of hybridization of a circularized probe on the first RCA product or intermediate molecule is important for the working of the method of the invention. The target-complementary region at each end of the probe is 6-16 nucleotides in length. A minimum size of at least 6 is selected to ensure specificity of binding, and the maximum size should not exceed 16 to allow for efficient RCA of the circularized probe, which is catenated to its target. Preferably the size range is 6-15, 6-14, 6-13 or 6-12 nucleotides, more particularly, 7-16, 7-15, 7-14, 7-13, 7-12, 8-16, 8-15, 8-14, 8-13, 8-12. In particular embodiments, a size of less than or up to 13 or 12 or 11 or 10 is preferred. Thus in preferred embodiments the size range is 6 or 7 to 8, 9, 10, 11 or 12, e.g. 6-11, 6-10, 6-9, 6-8, or 7-11, 7-10, 7-9, 7-8, or 8-11, 8-10, 8-9. The length of each target-complementary region of a probe may be the same or different (as long as it is within the above ranges). For example a probe may have target-complementary regions of 6+6, 6+7, 7+7, 6+8, 7+8, 8+8, 8+9, 7+9, 9+9, 10+9, 10+10, 10+11, 11+11, 11+12, 12+12 etc., i.e. any combination of any integers within the above-noted ranges.

The total length of the hybridized region may be 12-32, more particularly 12-30, 12-28, 12-26, 12-24, 12-23, 12-22, 12-21, 12-20, 12-19, 12-18, 12-17, 12-16, 13-30, 13-28, 13-26, 13-24, 13-23, 13-22, 13-21, 13-20, 13-19, 13-18, 13-17, 13-16, 14-30, 14-28, 14-26, 14-24, 12-23, 14-22, 14-21, 14-20, 12-19, 14-18, 14-17, 14-16, 15-30, 15-28, 15-26, 15-24, 15-23, 15-22, 15-21, 15-20, 15-19, 15-18, 15-17, or 15-16.

More particularly, the total length of the hybridized region is no more than 30, 28, 27, 26, 25, 24, 23, 22, 21, 20, 19, 18, 17 or 16 nucleotides.

As explained above, the hybridized region may in some embodiments be made up not only of the target-complementary regions of the probe, but may include also either a gap oligonucleotide or other gap-filled sequence (e.g. a gap sequence generated by extension of a 3′ end of the probe). Thus where the ligation of the probe ends is indirect, the free ends hybridise to the target with a space in between which is filled by a “gap” oligonucleotide such that each free end is ligated to one end of the gap oligonucleotide. Alternatively, the space in between the free ends may be “filled-in” by extending the free 3′ end, e.g. in a polymerase reaction, using the ligation template as an extension template. Once the free 3′ end has been extended to be adjacent to the free 5′ end, the two ends may be joined by a ligation reaction. Accordingly, the total hybridized regions mentioned above may be made up of the probe target complementary regions and optionally the gap-fill sequences.

It will be understood therefore that in the gap-fill embodiments of the invention where the probe ends are ligated indirectly to one another, the gap may be from 1-20 nucleotides long, e.g. 1-19, 1-18, 1-15, 1-12, 1-10, 1-8, 1-7, 1-6, 1-5, 1-4, or from 2, 3, 4, 5, or 6 to any of the upper limits of the above-noted ranges.

As shown in the Examples below, good results have particularly been obtained with padlock probes having target complementary regions 12, 10 and 8 nucleotides long and accordingly, it is preferred that the target complementary regions are up to 12, 11, 10, 9 or 8 nucleotides long, or that the length of the hybridized region is up to 24, 23, 22, 21, 20, 19, 18, 17 or 16 nucleotides long.

As generally discussed above, the methods of the invention allow a localised further RCA amplification of a first RCA, wherein a second or further RCA amplification product is localised to a first RCA amplification product. This can have a number of applications, most notably in signal amplification, and hence in any detection method or assay based on detecting an RCA product. Accordingly, in such an embodiment the methods of the invention as defined above may include an additional or further step of detecting a said attached second RCA product, thereby to detect said first RCA product, wherein detection is localised to the first RCA product.

Alternatively viewed, such an embodiment may be seen to provide a further aspect of the invention, which may be defined as a method for detection of a first product of an RCA reaction, said method comprising performing a localised RCA reaction as defined herein and detecting said second RCA product as defined above. In certain embodiments a localised detection (e.g. a spatial detection) may be allowed, for example where the first RCA product is immobilised or fixed in situ.

Advantages of such a method, as noted above, include stronger and/or faster signal amplification. The method thus has particular utility in the detection of any desired assay target or analyte, which may be a target/analyte nucleic acid molecule which may itself by amplified by RCA to form a first RCA product or a target/analyte nucleic acid molecule or any other molecule which may be detected by an assay which uses or generates a circular nucleic acid molecule as an assay reporter or a marker for the assay target/analyte (see above). Such a circle may be the first RCA template used to generate the first RCA product.

Thus the methods and probes of the invention may find utility in the detection of a nucleic acid molecule in a sample. The nucleic acid molecule may be the target analyte for detection or may be indicative of the presence of the target analyte in a sample. For instance, the nucleic acid molecule may be attached to the target, e.g. a nucleic acid domain of an antibody:nucleic acid conjugate which is bound, directly or indirectly, to the target, e.g. a protein molecule. Similarly, the nucleic acid molecule to be detected may be a nucleic acid molecule generated from the interaction between proximity probes, which are bound to the target analyte, e.g. a protein.

Accordingly, the invention may be seen to provide a method for detecting an analyte in a sample, wherein a first circular RCA template is used or generated (e.g. generated from a nucleic acid analyte or used or generated as a marker for said analyte), a first RCA reaction is performed using said first RCA template to generate a first RCA product, a localised second RCA reaction is performed as described herein to generate a second RCA product localised to said first RCA product, and said second RCA product is detected.

Thus a probe (circularisable oligonucleotide) as described herein may be used which hybridises directly or indirectly to a first RCA product. The first RCA product may be generated by a first RCA reaction using a first RCA template which may itself be or be derived or generated from:

(i) the analyte;

(ii) a nucleic acid molecule (e.g. probe) directly or indirectly attached to the analyte; or

(iii) indicative of, or a proxy for, (i.e. a marker for) the analyte in the sample.

RCA templates, i.e. circular or circularisable nucleic acid molecules, e.g. oligonucleotides, are well known in the art. A RCA template typically may comprise about 20-1000 nucleotides, e.g. 26-1000, 30-1000, 30-900, 60-900, 40-800, 50-700, 60-600, 70-500, 80-400, 90-300 or 100-200 nucleotides, such as at least 20, 25, 26, 27, 28, 29, 30, 35, 40, 50, 60, 70, 80, 90, 100, 120, 150, 200 or 250 nucleotides. More particularly in the methods of the invention the probe may 20-150, e.g. 20-120, 20-100, 25-150, 25-120, 30-150, 30-120, 30-100, 40-150, 40-120, 40-100 nucleotides.

The first and/or second RCA template may comprise a reporter domain, which is a sequence that can be used to detect and/or identify the RCA product, i.e. the primer extension product templated by the RCA template. This is particularly advantageous in multiplex embodiments of the invention, where more than one different first RCA product is subjected to the method e.g. where more than one analyte, e.g. nucleic acid analyte, is detected in a single assay. The second RCA template (i.e. the RCA template generated from a padlock probe) (e.g. each probe specific for a first RCA product derived from or indicative of a target analyte), may comprise a unique “marker” or identification sequence (e.g. a bar-code sequence, such as a site comprising the sequence of a specific detection probe, i.e. the RCA product is complementary to the RCA template and as such detection probes that hybridize to the RCA product will comprise a sequence that is identical to part of the RCA template) to allow the separate detection and/or quantification of each analyte in the sample. Thus, in multiplex assays each probe (or second RCA template) may comprise a different reporter domain and the detection of the interaction of the probe and the target analyte, i.e. the detection of each analyte, may be detected in parallel (i.e. at the same time), e.g. using oligonucleotides tagged with distinct fluorophores that may hybridise to the complement of the reporter domain. Alternatively, each marker (and therefore each analyte) may be detected using sequential visualisation reactions, wherein each reaction is separated by, e.g. stripping or bleaching steps. Methods of sequential visualisation reactions suitable for using the methods of the invention are known in the art, e.g. Göransson et al., 2009 (A single molecule array for digital targeted molecular analyses.Nucleic Acids Res. 2009 January; 37(1):e7), Wählby et al., 2002 (Sequential immunofluorescence staining and image analysis for detection of large numbers of antigens in individual cell nuclei.Cytometry, 47(1):32-41, 2002), which are hereby incorporated by reference. In some representative embodiments of the invention, multiple analytes may be detected in parallel. In other representative embodiments of the invention, multiple analytes may be detected sequentially.

Combinatorial methods of labelling, e.g. ratio labelling, using different combinations and/or ratios of different labels are known in the art and may be used to increase the number of different molecules, and hence different analytes which may detected at one time, or in the same reaction. For example, combinations using different coloured and/or fluorescent labels and/or different ratios of different coloured and/or fluorescent labels may be used. For example, such “colour”-coding with different combinations of coloured and/or fluorescent labels may be used in multiplex assays based on detection by flow cytometry or microscopy. Alternatively, using lanthanide isotope labels cyToF detection may be used. By way of example, 7 different fluorophores may be grouped into 4 different types. There are 7 different combinations if labelled with only one colour, with 2 colours there are 21 different combinations, for 3 and 4 colours there are 35 different combinations and so on.

The primer for the second RCA comprises a region of complementarity (defined further below) to a part of the second RCA template, which forms a duplex that is sufficiently stable under the conditions of the assay to facilitate RCA template dependent extension of the primer. The primer will generally be at least 5 nucleotides in length, typically at least 6, 8 or 10, usually at least 15 or 16 nucleotides in length and may be as long as 30 nucleotides in length or longer, where the length of the primer will generally range from 5 to 50 nucleotides in length, e.g. from 6, 8 or 10 to 50, 40, 30 or 20, usually from about 10 to 35 nucleotides in length.

As noted above, one or more intermediate molecules may be used, for example an intermediate molecule may comprise binding sites for a multiplicity of probes, or an intermediate molecule may comprise one binding site for a probe, and one intermediate molecule for each probe may be used (for example where the intermediate molecule binds to a site present in each monomer). For simplicity, the invention is defined primarily with respect to direct interactions between the probe and a first RCA product, but where reference is made to this in the context of probe binding, or to unwanted extension using the first RCA product as template it will be understood that this will apply analogously to binding to or extension on the intermediate molecule. Moreover, in the context of detection assay methods, the first RCA product or intermediate molecule with which the probe interacts may be viewed as the target nucleic acid molecule for the probe, even though the objective of the method may be the detection of a nucleic acid molecule or other analyte with which the probe does not interact directly.

A region of complementarity to its target nucleic acid molecule refers to a portion of the probe that is capable of forming an intermolecular duplex with at least a region of the target nucleic acid molecule. Subject to the size ranges indicated above, the regions of complementarity to the target nucleic acid molecule will be sufficient to form a stable duplex in the assay conditions in which the probe finds utility.

“Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary” under the invention. Usually two sequences are sufficiently complementary when at least about 85% (preferably at least about 90%, and most preferably at least about 95%) of the nucleotides share base pair organization over a defined length of the molecule.

The probe/circularisble oligonucleotide may be made up of ribonucleotides and/or deoxyribonucleotides as well as synthetic nucleotide residues that are capable of participating in Watson-Crick type or analogous base pair interactions. Thus, the nucleic acid domains may be DNA and/or RNA or any modification thereof e.g. PNA or other derivatives containing non-nucleotide backbones. In some embodiments, the probe may comprise an exonuclease block, such that it cannot be used as a primer in a target templated nucleic acid extension reaction, i.e. cannot be recognised as a primer by a polymerase enzyme and/or cannot be degraded to produce a nucleic acid molecule capable of priming extension of the target nucleic acid molecule.

As described above, the methods and probes of the invention may be useful for the detection of any target analyte, wherein if the target analyte is not a nucleic acid molecule, a first RCA template (or indeed a first RCA product) may be viewed as a marker for the analyte.

The “analyte”, or ultimate detection assay target or objective, may be any substance (e.g. molecule) or entity it is desired to detect. The analyte is thus the “target” of a detection method or use of the invention. The analyte may accordingly be any biomolecule or chemical compound it may be desired to detect, for example a peptide or protein, or nucleic acid molecule or a small molecule, including organic and inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for an nucleic acid analyte) and may lead directly to the generation of a first RCA template (e.g. a padlock probe). Alternatively, as discussed above, the specific binding partner may be coupled to a nucleic acid, which may be detected using an RCA strategy, e.g. in an assay which uses or generates a circular nucleic acid molecule which can be the first RCA template. Analytes of particular interest may thus include nucleic acid molecules, such as DNA (e.g. genomic DNA, mitochondrial DNA, plastid DNA, viral DNA etc) and RNA (e.g. mRNA, microRNA, rRNA, snRNA, viral RNA etc), and synthetic and/or modified nucleic acid molecules, (e.g. including nucleic acid domains comprising or consisting of synthetic or modified nucleotides such as LNA, PNA, morpholino etc), proteinaceous molecules such as peptides, polypeptides, proteins or prions or any molecule which includes a protein or polypeptide component, etc., or fragments thereof. The analyte may be a single molecule or a complex that contains two or more molecular subunits, e.g. including but not limited to protein-DNA complexes, which may or may not be covalently bound to one another, and which may be the same or different. Thus in addition to cells or microrganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA. Advantageously, where the analyte is a nucleic acid molecule, the nucleic acid may be detected in situ, i.e. without removing or extracting the nucleic acid from the cell. However, isolated and amplified nucleic acid molecules also represent appropriate target analytes.

All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way e.g. for storage.

Representative samples thus include any material which may contain a biomolecule, or any other desired or target analyte, including for example foods and allied products, clinical and environmental samples. The sample may be a biological sample, which may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa etc. Representative samples thus include whole blood and blood-derived products such as plasma, serum and buffy coat, blood cells, urine, faeces, cerebrospinal fluid or any other body fluids (e.g. respiratory secretions, saliva, milk, etc), tissues, biopsies, cell cultures, cell suspensions, conditioned media or other samples of cell culture constituents, etc. The sample may be pre-treated in any convenient or desired way to prepare for use in the methods and uses of the invention, for example by cell lysis or purification, isolation of the analyte, etc.

The detection of the target analyte depends upon the presence of an analyte in a sample, which leads to the generation of the first RCA product. A second RCA product is then generated according to the invention, and can be detected in order to detect the analyte. As discussed above, the second RCA product can lead to a much stronger and/or faster signal. In some embodiments it can be advantageous to detect both the first and second RCA products, e.g. using separate labelled detection probes for each. Thus detecting the second product, which has a stronger signal can allow detection at low magnification for example. A more precise localisation at higher magnification can further be obtained by detecting the first product.

Thus, upon the addition of appropriate polymerase and ligase enzymes, the presence of analyte in the sample may be detected by rolling circle amplification (RCA) of the second RCA template, i.e. by detecting the second RCA product, and optionally the first RCA product. The concatemeric RCA products provide the “signal” for detection of the analyte. Said signal may be detected by any appropriate means known in the art (see below for further examples) and as taught in U.S. Pat. No. 7,320,860, e.g. by hybridisation of labelled probes to a reporter domain sequence, which is repeated throughout the concatemeric RCA products.

Where ligation of the probe to form the second RCA template utilise a gap oligonucleotide, this may be provided separately. Hence, in the methods of the invention, a gap oligonucleotide may be added to the sample before, after, or contemporaneously with the probe. In some embodiments, several different gap oligonucleotides may be added, wherein each type of gap oligonucleotide is added at a different concentration. Each type of gap oligonucleotide may comprise common sequences at the 5′ and 3′ ends that are complementary to the ligation template domain (in between the ends of the circularisable oligonucleotide) and a different intervening sequence, which may act as a reporter domain as defined above. The resultant RCA products will comprise different reporter domain sequences depending on which gap oligonucleotide was ligated into the RCA template and can be detected separately. This may be utilised to extend the dynamic range of the assay methods described herein, as described in WO2012/049316.

The term “detecting” is used broadly herein to include any means of determining the presence of the analyte (i.e. if it is present or not) or any form of measurement of the analyte. Thus “detecting” may include determining, measuring, assessing or assaying the presence or absence or amount or location of analyte in any way. Quantitative and qualitative determinations, measurements or assessments are included, including semi-quantitative. Such determinations, measurements or assessments may be relative, for example when two or more different analytes in a sample are being detected, or absolute. As such, the term “quantifying” when used in the context of quantifying a target analyte(s) in a sample can refer to absolute or to relative quantification. Absolute quantification may be accomplished by inclusion of known concentration(s) of one or more control analytes and/or referencing the detected level of the target analyte with known control analytes (e.g. through generation of a standard curve). Alternatively, relative quantification can be accomplished by comparison of detected levels or amounts between two or more different target analytes to provide a relative quantification of each of the two or more different analytes, i.e., relative to each other.

The sequences of the probes may be chosen or selected with respect to the sequence of the probe and its target nucleic acid molecule. Thus, whilst the target-complementary regions are chosen with respect to the target molecule to which they bind, the sequence of the rest of the probe is not critical, but must of course include a binding site for the second RCA primer. However, the sequences should be chosen to avoid the occurrence of intramolecular hybridization. Once the sequence is selected or identified, the probe may be synthesized using any convenient method.

The term “hybridisation” or “hybridises” as used herein refers to the formation of a duplex between nucleotide sequences which are sufficiently complementary to form duplexes via Watson-Crick base pairing. Two nucleotide sequences are “complementary” to one another when those molecules share base pair organization homology. Hence, a region of complementarity in a domain of a probe refers to a portion of that domain that is capable of forming an intra- or intermolecular duplex, i.e. either a duplex within the same molecule (a hairpin structure) or a duplex with a different molecule or a different strand of the probe construct. These terms are also used to refer to base pair interactions which are analogous to Watson-Crick base pairing, including Hoogsteen base pairing which is a rarely observed variation of base pairing which also allows for a third strand to wind around a double-helix assembled in a Watson-Crick pattern to form a triplex.

The amount of probe that is added to a sample may be selected to provide a sufficiently low concentration of probe in the reaction mixture to minimise non-target specific interactions, i.e. to ensure that the probe will not randomly bind to non-target molecules in the sample to any great or substantial degree. For example, it is intended that only when the probe binds its target nucleic acid molecule (i.e. only in the presence of the first RCA product) is the second RCA template generated and allowed to generate a second RCA product. In representative embodiments, the concentration of each probe in the reaction mixture following combination with the sample containing the first RCA product ranges from about 1 fM to 1 μM, such as from about 1 pM to about 1 nM, including from about 1 pM to about 100 nM, e.g. 1, 2, 5, 10, 20, 50 nM.

A number of different probes may be added to a sample for a multiplex assay (for example in a situation where multiple different first RCA products or first RCA templates have been generated or added, in order to detect multiple analytes in parallel. Multiplex assays may involve the detection of tens, hundreds, thousands or even tens of thousands of analytes in a sample. Accordingly, multiplex assays may comprise at least 2 distinct probes, i.e. probes capable of hybridising (directly or indirectly) to different first RCA products and hence, for example, detecting different analytes. For instance, multiplex assays may utilise at least 3, 4, 5, 10, 20, 30, 40 or 50 probes, such as 100, 200, 500, 1000, 10000 or more probes.

Following combination of the sample (or reaction mixture) containing the first RCA product and the probe(s), the reaction mixture may be incubated for a period of time sufficient for the probe(s) to bind to its target (first RCA product, or intermediate molecule), if present, in the sample. As described above, once the probe has bound (directly or indirectly) to the first RCA product, the probe is ligated to generate the second RCA template and in one embodiment the second RCA primer may then be added and allowed to interact with the second RCA template so as to form a primer/RCA template complex for the second RCA. Once a primer/RCA template complex has formed, the primer may be extended using the second RCA template as a template for polymerisation. In some representative embodiments, e.g. in situ assays or other assays in which the first RCA product is immobilised, wash steps may be included between the addition of probe and the detection of the RCA product, e.g. the first RCA product may be captured or immobilised on a solid support or substrate, which may be washed to remove unbound or non-specifically bound probe or RCA products that are not attached to the first RCA product. In some embodiments, wash steps may be included between ligating the probe and the detection of second RCA product, to remove unligated probes. In other representative embodiments a cleaning step may be included before the detection of the RCA product. For example. a component having 3′ exonuclease activity may be added to the sample between ligating the probe and addition of the primer for the second RCA reaction to remove (digest or degrade) circularisable oligonucleotides which have not been circularised (unligated probes).

In representative embodiments, the probe and sample may be pre-incubated for a period of time ranging from 5 minutes to about 24 hours prior to the addition of the additional probes. Preferably said pre-incubation is from about 20 minutes to 12 hours at a temperature ranging from 4 to about 50° C. e.g. 10-40° C. or 20-37° C. Conditions under which the reaction mixture is maintained should be optimized to promote specific binding of the probe to its target nucleic acid molecule, while suppressing unspecific interaction.

Following the combination of the sample with the probe, the gap oligonucleotide(s) may be added, if used, and allowed to hybridise. Alternatively or additionally, one or more gap oligonucleotides may be added with the probe.

In general, any convenient protocol that is capable of detecting the presence of an RCA product may be employed to detect the second RCA product, and optionally the first RCA product. The detection protocol may or may not require a separation step.

As is known in the art, in template-directed ligation ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary (i.e. a ligation template). Any convenient ligase may be employed, where representative ligases of interest include, but are not limited to: Temperature sensitive and thermostable ligases. Temperature sensitive ligases include, but are not limited to, bacteriophage T4 DNA ligase, bacteriophage T7 ligase, and E. coli ligase. Thermostable ligases include, but are not limited to, Taq ligase, Tth ligase, Ampligase® and Pfu ligase. Thermostable ligase may be obtained from thermophilic or hyperthermophilic organisms, including but not limited to, prokaryotic, eukaryotic, or archael organisms. Certain RNA ligases may also be employed in the methods of the invention.

A suitable ligase and any reagents that are necessary and/or desirable may be combined with the reaction mixture and maintained under conditions sufficient for ligation of the hybridized oligonucleotides to occur. Ligation reaction conditions are well known to those of skill in the art. During ligation, the reaction mixture in certain embodiments may be maintained at a temperature ranging from about 4° C. to about 105° C., about 4 to about 80° C., such as about 10 to about 70° C., about 15 to about 60° C., typically such as from about 20° C. to about 37° C. for a period of time ranging from about 5 seconds to about 16 hours, such as from about 1 minute to about 1 hour. In yet other embodiments, the reaction mixture may be maintained at a temperature ranging from about 35° C. to about 45° C., such as from about 37° C. to about 42° C., e.g., at or about 38° C., 39° C., 40° C. or 41° C., for a period of time ranging from about 5 seconds to about 16 hours, such as from about 1 minute to about 1 hour, including from about 2 minutes to about 8 hours. In a representative embodiment, the ligation reaction mixture includes 50 mM Tris pH7.5, 10 mM MgCl₂, 10 mM DTT, 1 mM ATP, 25 mg/ml BSA, 0.25 units/ml RNase inhibitor, and T4 DNA ligase at 0.125 units/ml. In yet another representative embodiment, 2.125 mM magnesium ion, 0.2 units/ml RNase inhibitor; and 0.125 units/ml DNA ligase are employed.

It will be evident that the ligation conditions may depend on the ligase enzyme used in the methods of the invention. Hence, the above-described ligation conditions are merely a representative example and the parameters may be varied according to well known protocols. For example, a ligase that may be utilized in the methods of the invention, namely Ampligase®, may be used at temperatures of greater than 50° C. However, it will be further understood that the alteration of one parameter, e.g. temperature, may require the modification of other conditions to ensure that other steps of the assay are not inhibited or disrupted, e.g. binding of the probe to the target nucleic acid molecule. Such manipulation of RCA assay methods is routine in the art.

The next step of the method following the ligation step is to generate the second RCA product, i.e. to extend the second RCA primer using the second RCA template in a polymerisation reaction. Rolling-circle amplification (RCA) is well known in the art, being described in Dean et al., 2001 (Rapid Amplification of Plasmid and Phage DNA Using Phi29 DNA Polymerase and Multiply-Primed Rolling Circle Amplification, Genome Research, 11, pp. 1095-1099), the disclosures of which are herein incorporated by reference. The RCA primer is employed in a primer extension reaction, i.e. the RCA primer is extended on the second RCA template to generate the second RCA product, being a single concatemeric product. The RCA primer will be of sufficient length, as described above, to provide for hybridization to the RCA template under annealing conditions (described in greater detail below).

In addition to the above nucleic acid components, the reaction mixture produced in the subject methods typically includes a polymerase, e.g. phi29 DNA polymerase and other components required for a DNA polymerase reaction as described below. The desired polymerase activity may be provided by one or more distinct polymerase enzymes. In some embodiment the polymerase has exonuclease activity, e.g. 5′ and/or 3′ exonuclease activity.

In preparing the reaction mixture of this step of the subject methods, the various constituent components may be combined in any convenient order. For example, all of the various constituent components may be combined at the same time to produce the reaction mixture.

The amplified products of the RCA reaction (namely the second RCA reaction, but also optionally the first RCA product) may be detected using any convenient protocol, where the particular protocol employed may detect the RCA products non-specifically or specifically, as described in greater detail below. For instance, the second RCA product may be detected directly, e.g. the concatemer may be cleaved to generate monomer which may be detect using gel electrophoresis, or more preferably by hybridizing labelled detection oligonucleotides that hybridize to the reporter domain in the RCA product. Alternatively, the RCA product may be detected indirectly, e.g. the product may be amplified by PCR and the amplification products may be detected.

Representative non-specific detection protocols of interest include protocols that employ signal producing systems that selectively detect single or double stranded DNA products, e.g., via intercalation. Representative detectable molecules that find use in such embodiments include fluorescent nucleic acid stains, such as phenanthridinium dyes, including monomers or homo- or heterodimers thereof, that give an enhanced fluorescence when complexed with nucleic acids. Examples of phenanthridinium dyes include ethidium homodimer, ethidium bromide, propidium iodide, and other alkyl-substituted phenanthridinium dyes. In another embodiment of the invention, the nucleic acid stain is or incorporates an acridine dye, or a homo- or heterodimer thereof, such as acridine orange, acridine homodimer, ethidium-acridine heterodimer, or 9-amino-6-chloro-2-methoxyacridine. In yet another embodiment of the invention, the nucleic acid stain is an indole or imidazole dye, such as Hoechst 33258, Hoechst 33342, Hoechst 34580 (BIOPROBES 34, Molecular Probes, Inc. Eugene, Oreg., (May 2000)) DAPI (4′,6-diamidino-2-phenylindole) or DIPI (4′,6-(diimidazolin-2-yl)-2-phenylindole). Other permitted nucleic acid stains include, but are not limited to, 7-aminoactinomycin D, hydroxystilbamidine, LDS 751, selected psoralens (furocoumarins), styryl dyes, metal complexes such as ruthenium complexes, and transition metal complexes (incorporating Tb³⁺ and Eu³⁺, for example). In certain embodiments of the invention, the nucleic acid stain is a cyanine dye or a homo- or heterodimer of a cyanine dye that gives an enhanced fluorescence when associated with nucleic acids. Any of the dyes described in U.S. Pat. No. 4,883,867 to Lee (1989), U.S. Pat. No. 5,582,977 to Yue et al. (1996), U.S. Pat. No. 5,321,130 to Yue et al. (1994), and U.S. Pat. No. 5,410,030 to Yue et al. (1995) (all four patents incorporated by reference) may be used, including nucleic acid stains commercially available under the trademarks TOTO, BOBO, POPO, YOYO, TO-PRO, BO-PRO, PO-PRO and YO-PRO from Molecular Probes, Inc., Eugene, Oreg. Any of the dyes described in U.S. Pat. No. 5,436,134 to Haugland et al. (1995), U.S. Pat. No. 5,658,751 to Yue et al. (1997), and U.S. Pat. No. 5,863,753 to Haugland et al. (1999) (all three patents incorporated by reference) may be used, including nucleic acid stains commercially available under the trademarks SYBR Green, EvaGreen, SYTO, SYTOX, PICOGREEN, OLIGREEN, and RIBOGREEN from Molecular Probes, Inc., Eugene, Oreg. In yet other embodiments of the invention, the nucleic acid stain is a monomeric, homodimeric or heterodimeric cyanine dye that incorporates an aza- or polyazabenzazolium heterocycle, such as an azabenzoxazole, azabenzimidazole, or azabenzothiazole, that gives an enhanced fluorescence when associated with nucleic acids, including nucleic acid stains commercially available under the trademarks SYTO, SYTOX, JOJO, JO-PRO, LOLO, LO-PRO from Molecular Probes, Inc., Eugene, Oreg.

In yet other embodiments, a signal producing system that is specific for the RCA product, as opposed to nucleic acid molecules in general, may be employed to detect the amplification. In these embodiments, the signal producing system may include a probe nucleic acid or oligonucleotide that specifically binds to a sequence found in the RCA product (i.e. a reporter domain sequence), where the probe nucleic acid/oligonucleotide may be labelled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, where directly detectable labels of interest include, but are not limited to: fluorescent labels, radioisotopic labels, chemiluminescent labels, and the like. In many embodiments, the label is a fluorescent label, where the labelling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labelled probe nucleic acids (i.e. detection probes) include, but are not limited to: fluorescein, the cyanine dyes, such as Cy3, Cy5, Alexa 555, Bodipy 630/650, and the like. Other labels, such as those described above, may also be employed as are known in the art.

In certain embodiments, the specifically labelled probe nucleic acids (detection probes) are labelled with “energy transfer” labels. As used herein, “energy transfer” refers to the process by which the fluorescence emission of a fluorescent group is altered by a fluorescence-modifying group. Energy transfer labels are well known in the art, and such labelled oligonucleotide probes include the TaqMan® type probes, as described in U.S. Pat. No. 6,248,526, the disclosure of which is herein incorporated by reference (as well as Held et al., Genome Res. (1996) 6:986-994; Holland et al., Proc. Natl Acad. Sci. USA (1991) 88:7276-7280; and Lee et al., Nuc. Acids Res. (1993) 21:3761-3766). Further examples of detection probes include: Scorpion probes (as described in Whitcombe et al., Nature Biotechnology (1999) 17:804-807; U.S. Pat. No. 6,326,145, the disclosure of which is herein incorporated by reference), Sunrise probes (as described in Nazarenko et al., Nuc. Acids Res. (1997) 25:2516-2521; U.S. Pat. No. 6,117,635, the disclosure of which is herein incorporated by reference), Molecular Beacons (Tyagi et al., Nature Biotechnology (1996) 14:303-308; U.S. Pat. No. 5,989,823, the disclosure of which is incorporated herein by reference), and conformationally assisted probes (as described in provisional application Ser. No. 60/138,376, the disclosure of which is herein incorporated by reference).

Thus, determining the presence of the second, and optionally first, RCA product may be achieved using any convenient protocol. The reaction mixture may be screened etc. (i.e., assayed, assessed, evaluated, tested, etc.) for the presence of any resultant second, and optionally first, RCA products in order to detect the presence of the target analyte in the sample being assayed. The particular detection protocol may vary depending on the sensitivity desired and the application in which the method is being practiced.

The RCA product may be detected in a number of different ways. For example, the nucleotides incorporated in the RCA product may be directly labelled, e.g., fluorescently, or otherwise spectrophotometrically, or radioisotopically labelled or with any signal-giving label, such that the RCA product is directly labelled. In some embodiments detection probes as discussed above, e.g., fluorescently labelled probes, molecular beacons (as described above) etc. may be employed to detect to the presence of the RCA product, where these probes are directed to a sequence (reporter domain sequence, i.e. a sequence that is identical to the reporter domain sequence in the RCA template) that is repeated in the RCA concatemer and therefore only exists in its entirety in the RCA product.

The reaction mixture prepared in this detection step of the subject methods may further include an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulphate, and the like may be employed. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, although higher or lower amounts may be used and may depend on the type of reaction. For instance, for PCR the amount of Mg²⁺ present in the buffer may be about 1.5 mM, whereas for RCA, the amount of Mg²⁺ present in the buffer may about 10 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, where most preferred is pH 7.3 at 72° C. Other agents which may be present in the buffer medium include chelating agents, such as EDTA, EGTA and the like.

The next step in the subject methods is signal detection from the labelled RCA products of interest, where signal detection may vary depending on the particular signal producing system employed. In certain embodiments, merely the presence or absence of detectable signal, e.g., fluorescence, is determined and used in the subject assays, e.g., to determine or identify the presence or absence of the second (and optionally first) RCA product (and hence the target analyte). Depending on the particular label employed, detection of a signal may indicate the presence or absence of the second (or first) RCA product.

In those embodiments where the signal producing system is a fluorescent signal producing system, signal detection typically includes detecting a change in a fluorescent signal from the reaction mixture to obtain an assay result. In other words, any modulation in the fluorescent signal generated by the reaction mixture is assessed. The change may be an increase or decrease in fluorescence, depending on the nature of the label employed, but in certain embodiments is an increase in fluorescence. The sample may be screened for an increase in fluorescence using any convenient means, e.g., a suitable fluorimeter, such as a thermostable-cuvette or plate-reader fluorimeter, or, for example where the sample is a tissue sample on a microscope slide, fluorescence may be detected using a fluorescence microscope. Fluorescence is suitably monitored using a known fluorimeter. The signals from these devices, for instance in the form of photo-multiplier voltages, are sent to a data processor board and converted into a spectrum associated with each sample tube. Multiple tubes, for example 96 tubes, can be assessed at the same time. Thus, in some embodiments multiple analytes may be detected in parallel, whereas in other embodiments multiple analytes may be detected sequentially, e.g. one analyte at a time or one group of analytes at a time.

Where the detection protocol is a real time protocol, e.g., as employed in real time PCR reaction protocols, data may be collected in this way at frequent intervals, for example once every 3 minutes, throughout the reaction. By monitoring the fluorescence of the reactive molecule from the sample during each cycle, the progress of the amplification reaction can be monitored in various ways. For example, the data provided by melting peaks can be analyzed, for example by calculating the area under the melting peaks and these data plotted against the number of cycles.

The spectra generated in this way can be resolved, for example, using “fits” of pre-selected fluorescent moieties such as dyes, to form peaks representative of each signalling moiety (i.e. fluorophore). The areas under the peaks can be determined which represents the intensity value for each signal, and if required, expressed as quotients of each other. The differential of signal intensities and/or ratios will allow changes in labelled probes to be recorded through the reaction or at different reaction conditions, such as temperatures. The changes are related to the binding phenomenon between the oligonucleotide probe and the target sequence or degradation of the oligonucleotide probe bound to the target sequence. The integral of the area under the differential peaks will allow intensity values for the label effects to be calculated.

Screening the mixture for a change in fluorescence provides one or more assay results, depending on whether the sample is screened once at the end of the primer extension reaction, or multiple times, e.g., after each cycle, of an amplification reaction (e.g., as is done in real time PCR monitoring).

The data generated as described above can be interpreted in various ways. In its simplest form, an increase or decrease in fluorescence from the sample in the course of or at the end of the amplification reaction is indicative of an increase in the amount of the target analyte present in the sample, e.g. as correlated to the amount of RCA product detected in the reaction mixture, suggestive of the fact that the amplification reaction has proceeded and therefore the target analyte was in fact present in the initial sample. Quantification is also possible by monitoring the amplification reaction throughout the amplification process. Quantification may also include assaying for one or more nucleic acid controls in the reaction mixture, as described above.

In this manner, a reaction mixture may readily be screened (or assessed or assayed etc.) for the presence of RCA product, and hence of target analyte(s), e.g. nucleic acid analytes. The methods are suitable for detection of a single target analyte as well as multiplex analyses, in which two or more different target analytes are assayed in the sample. In these latter multiplex situations, the number of different sets of probes that may be employed typically ranges from about 2 to about 20 or higher, e.g., as up to 100 or higher, 1000 or higher, etc. wherein the multiple analytes in a sample may be detected in parallel or sequentially.

The analysis of many analytes simultaneously and in a single reaction using several different probes (multiplexing) may enhanced by the increased sensitivity, and in certain embodiments also increased specificity, which may be obtained using the methods and probes of the invention. Each probe set can be designed to produce a RCA product that can be used to determine the presence or absence, quantity and/or location of the analytes ultimately being interrogated by the probe. The RCA product may be detected using any of the well established methods for analysis of nucleic acid molecules known from the literature including liquid chromatography, electrophoresis, mass spectrometry, microscopy, real-time PCR, fluorescent probes, microarray, colorimetric analysis such as ELISA, flow cytometry, mass spectrometry (CyTOF) etc.

The probes and methods of the present invention may be employed homogeneously (i.e. in solution) as described above, or alternatively heterogeneously, using a solid phase, for example, in which the first RCA product becomes immobilised on a solid phase, permitting the use of washing steps. This may result from immobilisation of the target analyte, for example in in situ detection procedures. The use of solid phase assays offers advantages, particularly for the detection of difficult samples: washing steps can assist in the removal of unbound and/or unligated probes etc, inhibiting components, and analytes can be enriched from an undesirably large sample volume. Higher concentrations and greater amounts of probes can be used, as unbound analytes, probes and RCA products can be removed by washing.

Immobilisation of the first RCA product and/or analyte on a solid phase may be achieved in various ways. Accordingly, several embodiments of solid phase assays are contemplated. In one such embodiment, the analyte can first be captured by an immobilised (or immobilisable) capture probes, the first RCA product can generated such that it is attached to the analyte, for example by virtue of the primer for the first RCA product being attached to the analyte, e.g. by being coupled to a binding partner for the analyte, and the first RCA product may then be bound by subsequently added probe(s) to generate the template for the second RCA. Alternatively, the first RCA product may simply be immobilised to a solid support. For example the primer for the first RCA product may be provided with an immobilisable group or moiety or means for immobilisation, or may be immobilised, prior to the first RCA.

The immobilised capture probe or first RCA primer, or first RCA product, may be immobilised, i.e. bound to the support, in any convenient way. Thus the manner or means of immobilisation and the solid support may be selected, according to choice, from any number of immobilisation means and solid supports as are widely known in the art and described in the literature. Thus, the capture probe, or first RCA primer or first RCA product may be directly bound to the support (e.g. chemically crosslinked), it may be bound indirectly by means of a linker group, or by an intermediary binding group(s) (e.g. by means of a biotin-streptavidin interaction). Thus, a capture probe or first RCA primer or first RCA product may be provided with means for immobilisation (e.g. an affinity binding partner, e.g. biotin or a hapten or a nucleic acid molecule, capable of binding to its binding partner, i.e. a cognate binding partner, e.g. streptavidin or an antibody or a nucleic acid molecule) provided on the support. A capture probe may be immobilised before or after binding to the analyte. Further, such an “immobilisable” capture probe may be contacted with the sample together with the support. Analogously, a first RCA primer may be immobilised before or after the first RCA etc.

The capture probe may be, for example, an antibody or nucleic acid molecule that is capable of binding to the target analyte specifically. In other words the capture probe may be an immobilised (or immobilisable) analyte-specific probe comprising an analyte binding domain (i.e. an analyte capture probe). Thus in such an embodiment the analyte is first captured by the immobilised or immobilisable capture probe which serves only to immobilise the analyte on the solid phase, and subsequently the immobilised analyte is subjected to a detection protocol which uses, or leads to the generation of, a first RCA template, wherein the primer for the first RCA is attached to the analyte. In such an embodiment, the capture probe may be any binding partner capable of binding the analyte, directly or indirectly. More particularly, such a capture probe binds specifically to the analyte.

The solid support may be any of the well known supports or matrices which are currently widely used or proposed for immobilisation, separation etc. These may take the form of particles (e.g. beads which may be magnetic or non-magnetic), sheets, gels, filters, membranes, fibres, capillaries, or microtitre strips, tubes, plates or wells etc.

The support may be made of glass, silica, latex or a polymeric material. Suitable are materials presenting a high surface area for binding of the analyte. Such supports may have an irregular surface and may be for example porous or particulate e.g. particles, fibres, webs, sinters or sieves. Particulate materials e.g. beads are useful due to their greater binding capacity, particularly polymeric beads.

Conveniently, a particulate solid support used according to the invention will comprise spherical beads. The size of the beads is not critical, but they may for example be of the order of diameter of at least 1 and preferably at least 2 μm, and have a maximum diameter of preferably not more than 10, and e.g. not more than 6 μm.

Monodisperse particles, that is those which are substantially uniform in size (e.g. size having a diameter standard deviation of less than 5%) have the advantage that they provide very uniform reproducibility of reaction. Representative monodisperse polymer particles may be produced by the technique described in U.S. Pat. No. 4,336,173.

However, to aid manipulation and separation, magnetic beads are advantageous. The term “magnetic” as used herein means that the support is capable of having a magnetic moment imparted to it when placed in a magnetic field, i.e. paramagnetic, and thus is displaceable under the action of that field. In other words, a support comprising magnetic particles may readily be removed by magnetic aggregation, which provides a quick, simple and efficient way of separating the particles following the analyte binding steps.

In a further embodiment, the analyte itself may be immobilised (or immobilisable) on the solid phase e.g. by non-specific absorption. In a particular such embodiment, the analyte may be present within cells, being optionally fixed and/or permeabilised, which are (capable of being) attached to a solid support, e.g. a tissue sample comprising analyte may be immobilised on a microscope slide.

The above-described methods typically result in detection of target dependent first RCA products (i.e. first RCA products that are only produced in the presence of the target analyte) that are present in the reaction mixture. This leads to the generation of second RCA products which in turn provides a measure of the amount of target analyte in the sample being assayed. The measure may be qualitative or quantitative.

Accordingly, the above described probes and methods for detecting the presence of one or more target analytes in a complex sample find use in a variety of different applications.

The subject probes and methods may be used to screen a sample for the presence or absence of one or more target analytes in a sample. As indicated above, the invention provides probes and methods for detecting the presence or quantifying the amount of one or more target analytes in a sample.

The subject probes and methods can be employed to detect the presence of one or more target analytes in a variety of different types of samples, including complex samples having large amounts of non-target entities. The subject methods are highly sensitive for detecting one or more target analytes in a simple or complex sample.

It will be evident from the description above and the representative examples described below that the methods and probes of the invention have advantages over existing methods. Notably, the methods allow for signal amplification of the signal from the first RCA product, thereby increasing the sensitivity of the method, and, as also noted above, faster signal generation. Increased sensitivity may permit analytes to be detected which are present only in low amounts. Essentially, the method permits an enhanced signal to be developed from an RCA reaction. A larger, more conspicuous reaction product is formed, which may more readily and easily be detected. The amplified signal from the second RCA product is localised to the first RCA product. This may permit highly sensitive localised detection of an analyte, for example in situ detection.

Thus in a method according to the present invention, a first RCA product may be generated in a highly specific manner, that is production of the first RCA product, or indeed first RCA template, may be strictly dependent upon the presence of an analyte (e.g. in the case of a padlock probe, or an assay using proximity probes which must both bind and interact to generate a circular RCA template). Advantageously, according to the method, the second RCA is dependent upon the presence of the first RCA product (as discussed above), but the requirement for specificity in this second RCA step is less strict (indeed it can be much less strict) and some background generation of second RCA product can be tolerated, as in the absence of first RCA product this will be far less than in its presence (both in terms of signal strength and size). In the presence of first RCA product, i.e. when a sRCA reaction takes place, a very strong signal amplification takes place. This is demonstrated in the Examples below.

Whilst not wishing to be bound by theory, it is believed that two or more generations of RCA product, when attached together, and labelled (e.g. with detection probes, for example for the second RCA product) will travel together as a single particle in a fluid. This may open up the possibility to use other detection modalities to detect the second RCA product (or the sRCA product of the method), for example flow cytometry or CyTOF, thereby broadening up the instrument base available for detection in RCA-based assays.

The strong signal amplification afforded by the second RCA reaction may allow ready and easy visualisation of signals, for example microscopically at low magnification or on a digitally scanned image and hence may permit rapid and easy visual inspection of assay results in a clinical scenario, e.g. inspection of pathology results in routine use. Thus the methods of the invention are particularly suited to clinical analysis procedures.

As noted above both first and second RCA products can be detected, allowing the second products to be easily detected e.g. at low magnification, whilst preserving the more precise location of the first RCA products using detection probes specific for the first RCA products.

This can for instance be helpful to identify rare integrated copies of viral genomes in human tissues or for otherwise detecting rare RCA products such as upon inefficient mutation detection in tissues. Another example when easy identification of a rare event may be helpful is when screening for the presence of circulating tumour cells (CTC) among a vast majority of non-CTC cells. The strong signal of the sRCA reaction allows fast and easy identification of events (detection of CTCs) at low magnification and the more precise localisation of the first RCA product visualised at higher magnification allows verification that the signal originated in a CTC and not in adjacent non-CTC cell.

The faster signal amplification which may be achieved by initiating the second RCA whilst the first is ongoing is also discussed above. Thus the methods allow RCA-dependent detection assays to be speeded up, which may be of value in at point of care locations such as doctor's offices etc. Further increases of speed and/or signal strength are possible by carrying out further RCA rounds or generations in the sRCA method.

The increases in signal strength/speed may allow other means of detection beyond the conventional fluorescence based methods, for example using turbidometric, magnetic, particle counting, electric, surface sensing, and weight-based detection techniques. For example one individual sRCA product from a second generation RCA after a 1 hour amplification has the potential weight of several femtograms. Such a weight increase may be detected by methods and means known in the art such as cantilevers, surface plasmon methods, and microbalances e.g. quartz crystal microbalances etc.

Since the method of present invention generates an enhanced signal which is localised to the product of the first RCA, it also confers the ability to count individual reaction products (second RCA products), triggered by individual nucleic acid circles (first RCA templates), using standard flow cytometers or distributed on a planar surface, etc. for highly precise digital detection. Thus, the method may permit an equivalent reaction to digital PCR, but with no need for emulsions or microfabricated structures, or finding conditions where exactly one template is present per compartment.

The prominent amplification products derived from the method of the present invention will further permit cloning of individual DNA circles, since the product obtained from an individual circle (e.g. first RCA template) may be visualised. An individual sRCA product may therefore be identified and isolated. For example, with the aid of the amplification method of the present invention visualization can be achieved in low melt agarose for isolation with no need for magnification, and the product may then be isolated e.g. scoped out with a toothpick, analogously to the isolation of bacterial colonies.

The sRCA methods of the present invention offer the potential to identify and count even very rare mutations, including when relatively error-prone mutation detection methods are used. Thus a first RCA product may be generated using a mutation-containing analyte target nucleic acid circle as first RCA template and the repeated occurrence of a mutant sequence in the first RCA product may be detected, by using an allele-specific (e.g. mutation-specific) probe for the second RCA i.e. a probe which recognises the mutant sequence present in the monomer repeats of the first RCA product.

The detection of rare mutations can be very important clinically for diagnosis. For example mutations in certain genes (e.g. KRAS mutations) can be diagnostically important and may serve to identify the emergence of acquired resistance to particular therapies (e.g. anti-EGFR therapy). Much effort has focused in recent years on developing methods for detecting such mutations. The method of the present invention could provide a useful addition to such methods.

An example of a method to detect rare mutations utilising the sRCA reaction of the present invention may involve isolating DNA from target cells, fragmenting the DNA, isolating a fragment which may contain a target sequence (e.g. mutation) of interest using a “selector” probe which is designed to have two target-specific ends which bind specifically to a target region of interest and allow the target fragment to be circularised, and performing a first RCA reaction using the circularised target fragment as first RCA template. The first RCA product thus generated may be subjected to the sRCA method of the present invention. The probe for the second RCA can be designed to recognise (e.g. bind to, and/or be ligated by) a mutant sequence it is desired to detect. Thus, by isolating target sequences to generate a first RCA template, the opportunity is afforded to generate a first RCA product comprising multiple (complementary) copies of the target sequence. Thus, an increased number of “targets” can be created in the concatemeric first RCA product. Each of these may be bound by a probe, which may be designed to be specific for the mutation it is desired to detect, to initiate a second RCA reaction, and the second RCA product can be detected to detect the mutation.

The invention will be further described with reference to the following non-limiting Examples with reference to the following drawings in which:

FIG. 1 depicts a representative padlock sRCA reaction according to the invention.

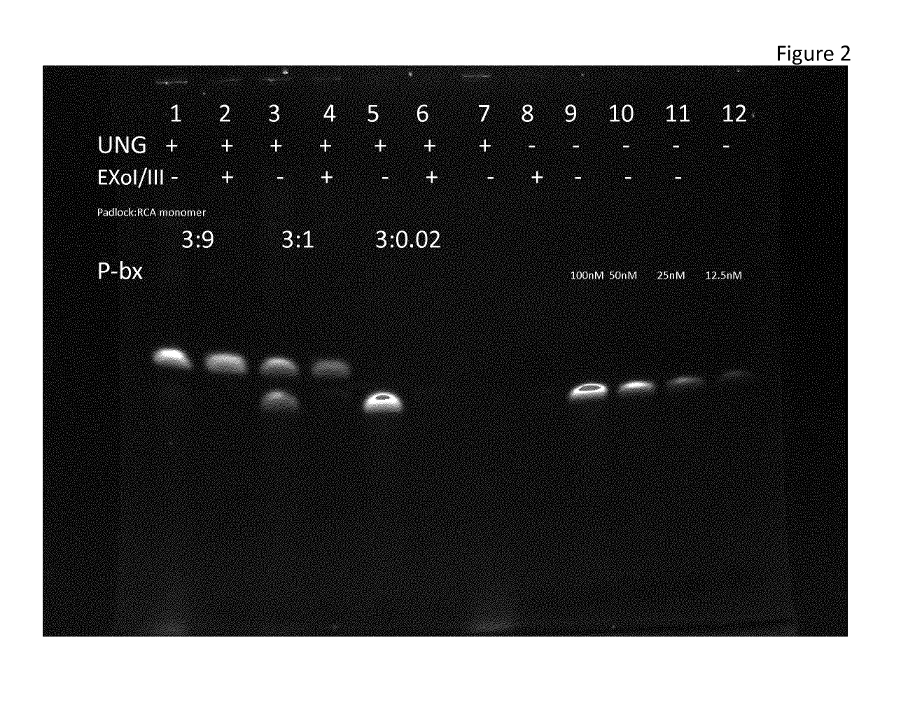

FIG. 2 shows the results of an experiment showing the effect of ligation efficiency on RCA products. The first RCA products were generated with dUTP, wells 1-7 have dUTP containing RCA products, and wells 1-6 have ligated padlock probes (secondary RCA probes). The padlock: RCA monomer ratio in wells 1-2 is 3:9, in wells 3-4 is 3:1 and in wells 5-6 is 3:0.02. The samples from wells 1-6 were treated either with UNG only or UNG Exol/III treated. UNG degrades only the RCA products while exol/III treatment degrades all single strand or double stand oligonucleotides and only the circularized DNA can stay intact. In well 8 there is only padlock, and after the Exol/III treatment, it was totally degraded. In wells 9-12 are only padlocks at different concentration as a concentration reference to estimate the ligation efficiency under different padlock: RCA monomer ratio.

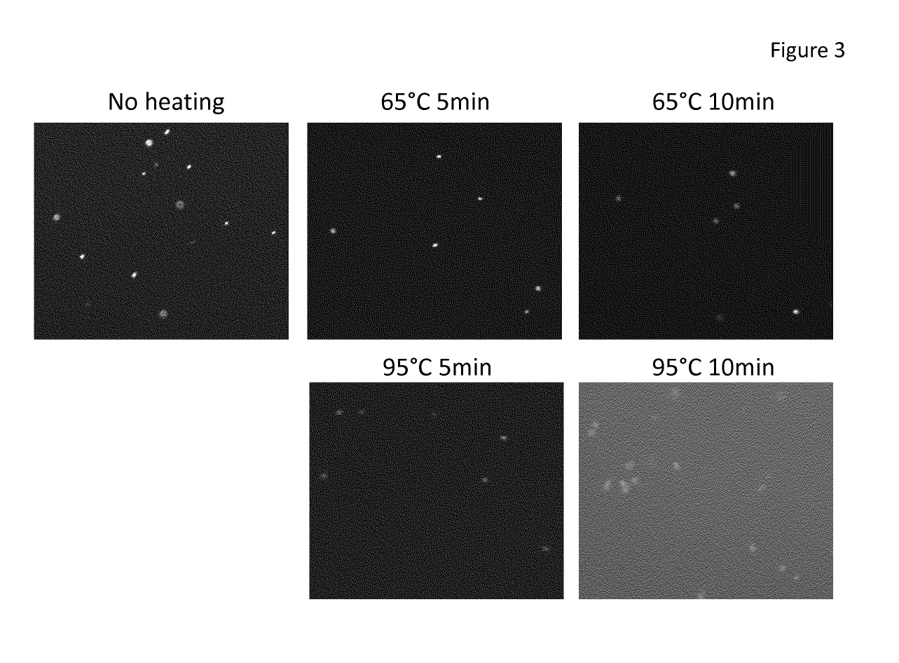

FIG. 3 shows the results of a stability test on padlock sRCA reaction products at different temperatures: no heating, 65° C. for 5 or 10 minutes, 95° C. for 5 or 10 minutes.

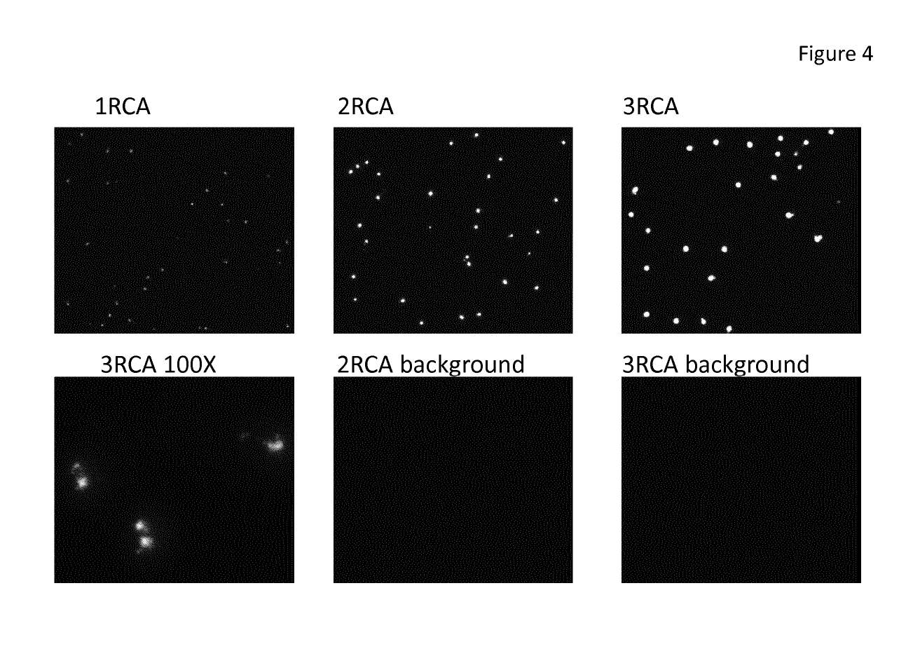

FIG. 4 shows the results from 3 rounds of RCA. The RCA reaction products were all labeled with Cy3 oligo and taken under the same exposure time 200 ms, except the 100× objective lens image, which was taken under 93 ms. The overall experiment is done as follows: 1RCA sample: rolling for 100 min; 2RCA sample: first rolling 20 min, first ligation 20 min, and the second rolling for 60 min 3RCA sample: first rolling 20 min, first ligation 20 min, and second rolling 20 min, second ligation 20 min, and third rolling for 20 min. The overall reaction times for all the samples were the same (100 min). The 2RCA background and 3RCA background are control experiments in which there were no first RCA template, and shows the RCA signals coming from the sRCA probes.

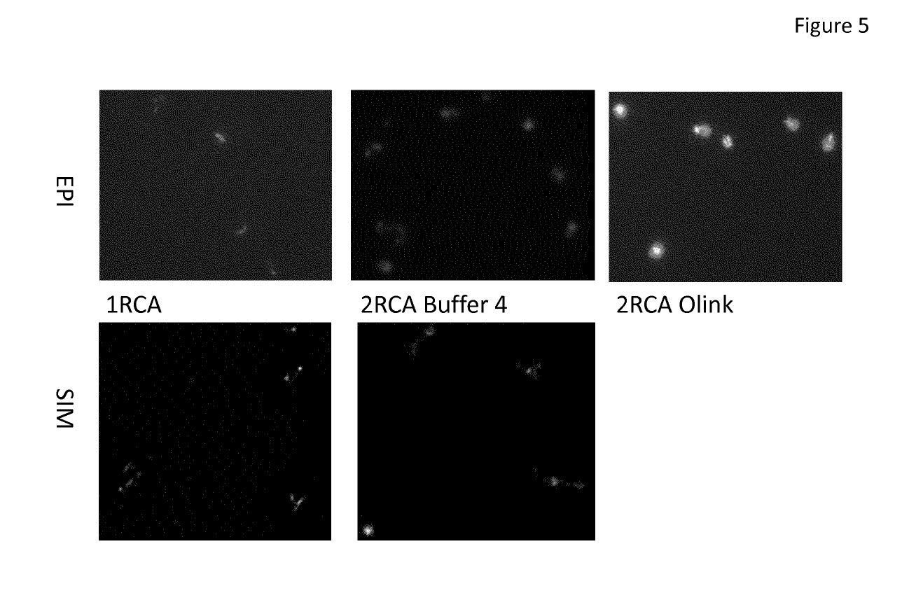

FIG. 5 compares results of a padlock sRCA reaction taken by either 100× EPI microscope or 100× SIM. And they are paired in vertical direction.

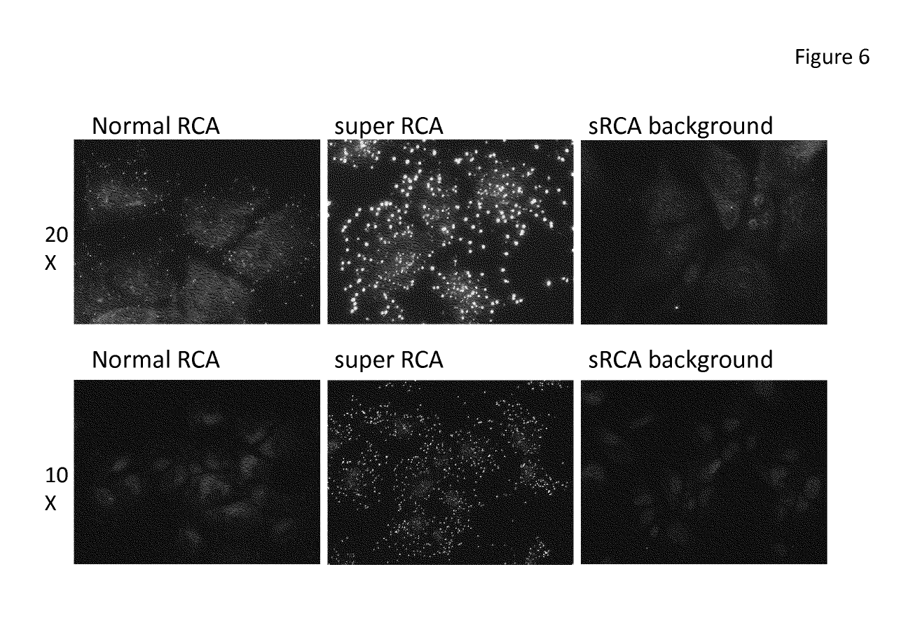

FIG. 6 shows the results of an experiment using padlock sRCA to detect Lgr5 target antigen in situ in a Hs578T breast cancer cell line with a proximity ligation assay PLA). In situ padlock probes (secondary probes): Rabbit+/−, against the same primary Lgr5 antibody This first rolling padlock has two ligation sites. In the normal RCA and sRCA pictures both plus and minus PLA probes were applied, but in the sRCA background pictures, only the Plus probes were applied. In the rolling settings, the first RCA step took 1 hr and followed by 30 min ligation in the sRCA and sRCA background well, during the ligation step, the normal RCA well were incubated with only ligation buffer. After the RCA-ligation step, the RCA reaction was allowed to roll for another 1 hr. The normal RCA and super RCA were detected with the same detection oligo sequence and fluorphore (Cy3). All the six images were taken under the same exposure time, for the DAPI the exposure time is 90 ms, and the exposure time for Cy3 channel is 1500 ms. The top three images were taken at 20× objective lens, and the lower three images were taken under 10× objective lens. The two images (10× and 20×) from each experiment setting (normal RCA, sRCA, sRCA background) were taken at the same position.

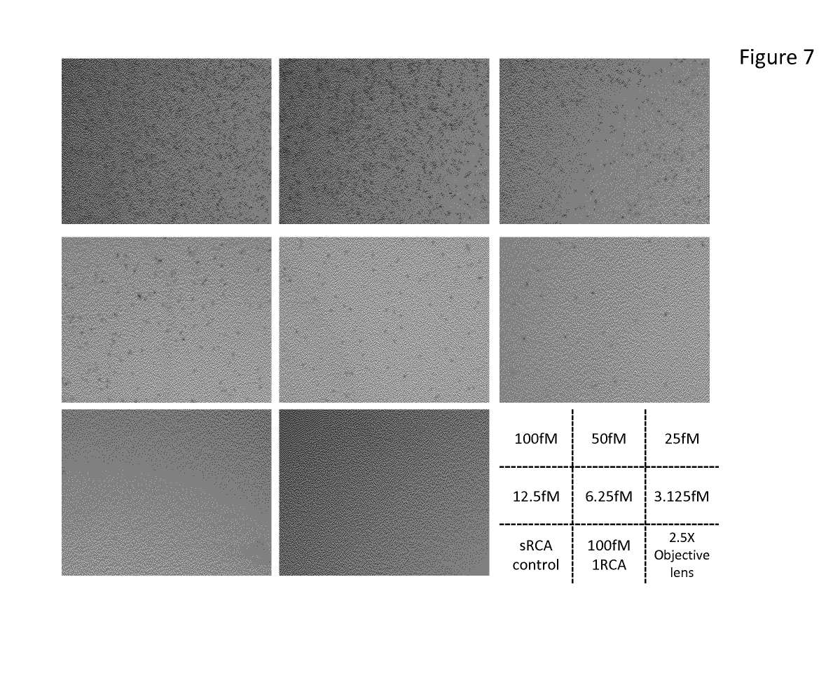

FIG. 7 shows the results of a padlock-sRCA reaction performed on slides and detected using a HRP readout. The image was taken by bright field microscopy using a 2.5× objective lens.

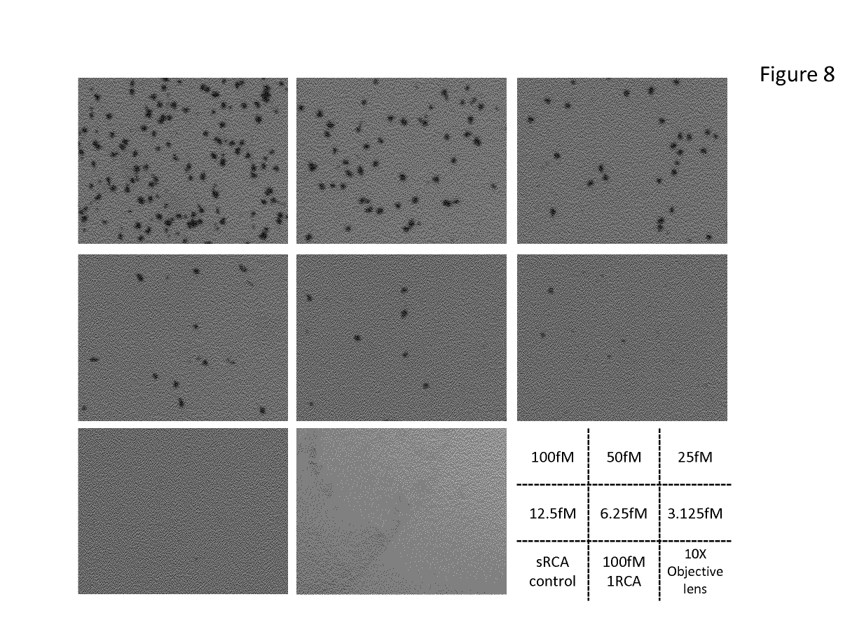

FIG. 8 shows the results of a padlock-sRCA reaction performed on slides and detected using a HRP readout. The image was taken by bright field microscopy using a 10× objective lens.

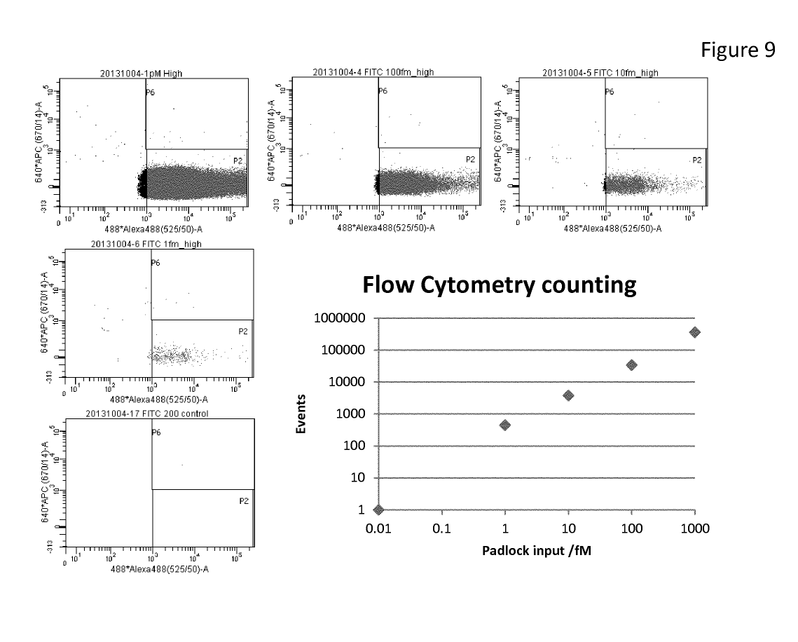

FIG. 9 presents dot plots showing detection of padlock sRCA products by flow cytometry at different concentrations of first RCA padlock 1 pM, 100 fM, 10 fM, and 1 fM.

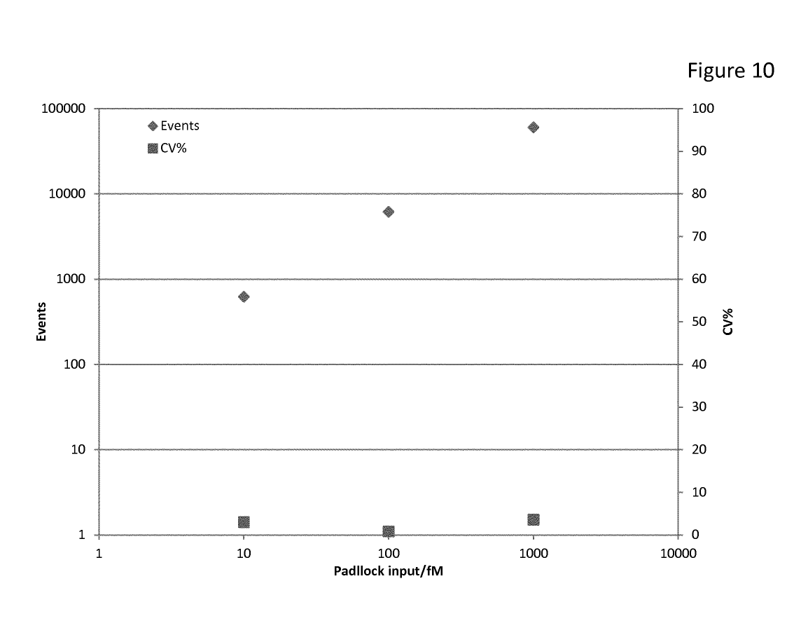

FIG. 10 is a graph showing coefficient of variation from the flow cytometry experiment in Example 7. Diamonds show events at different input padlock concentrations (shown in fM) calculated as the average number from 3 different counts. Squares show the coefficient of variation (CV) calculated based on 3 counts.

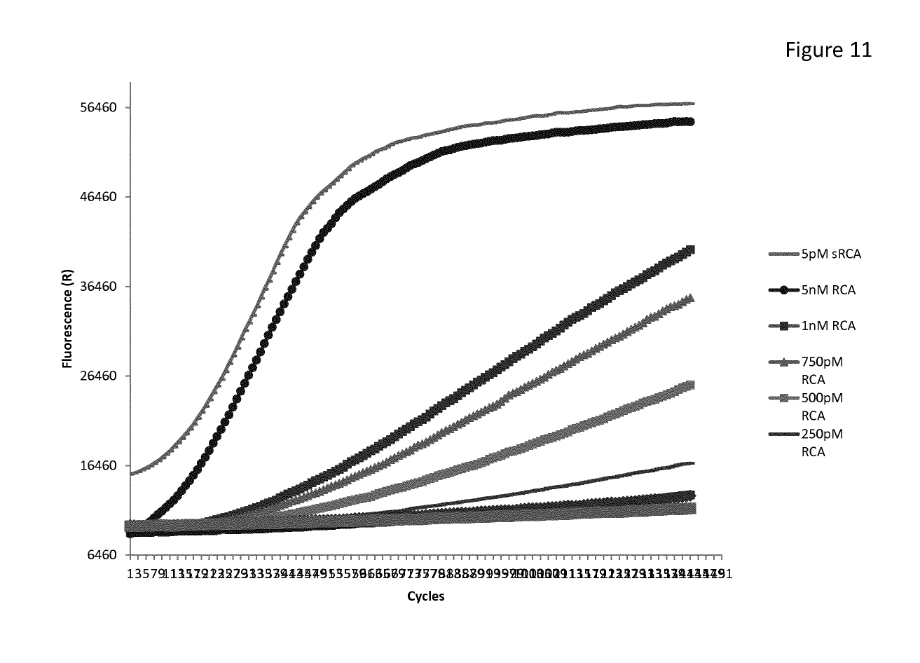

FIG. 11 is an amplification plot showing RCA growth curves (fluorescence vs cycle number) comparing a padlock sRCA reaction (straight line curve) to a normal RCA (one round only) with different padlock concentrations (5 nM, 1 nM, 750 pM, 500 pM, 250 pM, 125 pM, 75 pM and 50 pM). 5 pM of the first RCA template was rolled at 37 degrees for 60 minutes. The size of the first padlock is 87 bp, and on the assumption that the rolling speed for the phi29 enzyme at 37 degree was at 2000 base per minute, there are around 1379 repeats of the padlock minus strand. Then the first RCA product was ligated with 100 nM second RCA padlock for 30 minutes at 37 degree. After that, sybr and rolling mix were added and put into the PCR machine to measure the fluorescent value every one minute.

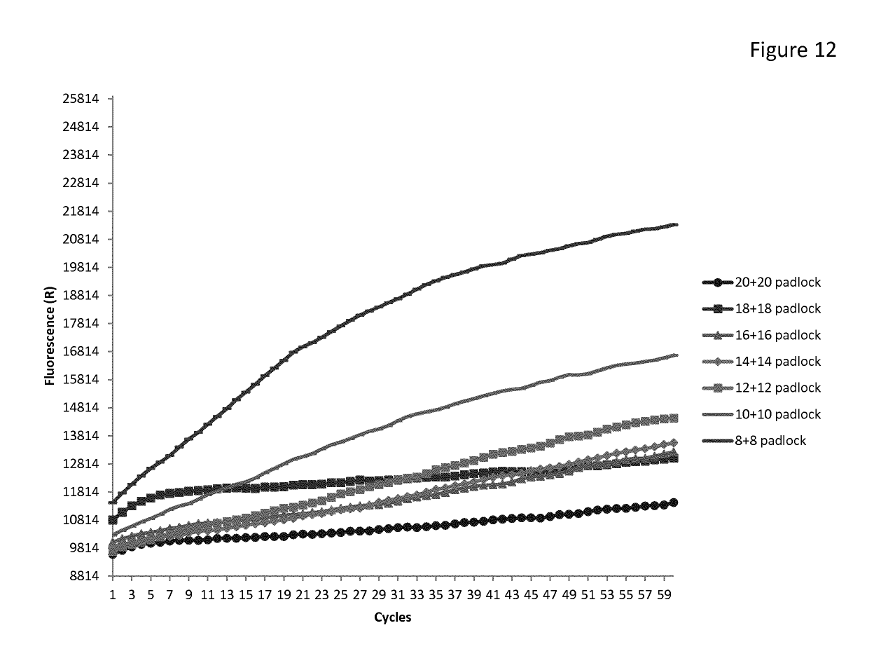

FIG. 12 is graph comparing rolling efficiency in padlock sRCA reactions of padlocks with different lengths of target complementary regions (8+8, 10+10, 12+12, 14+14, 16+16, 18+18 and 20+20 nucleotides) which bind to the first RCA product. A series of 90 bp long padlocks was used, having the same backbone and ligation site, the difference between the padlocks being the arm length for the hybridization. The longest arm is 20+20, and for the shorter arms the border bases were mutated to unmatched bases. The first RCA products were pre rolled for 1 hr and then the second RCA padlock was ligated on the RCA products for 1 hr. Then the rolling mix and sybr Gold were added into the ligation mix and put into the Q-PCR machine to monitor the growth curve of each reaction. The data was collected every 1 min and lasted for 4 hrs.

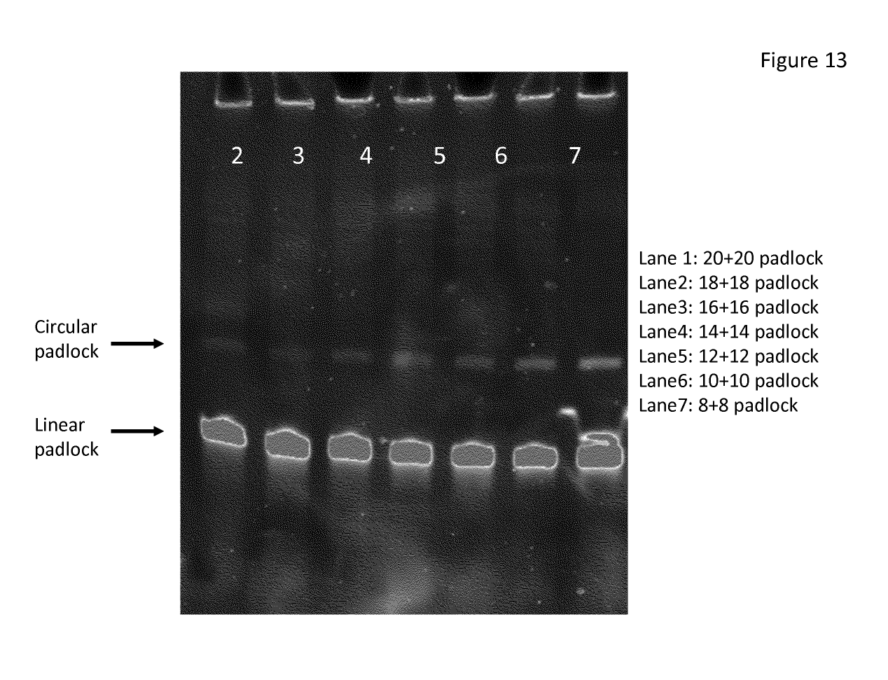

FIG. 13 presents a gel image showing the ligation efficiency of the secondary padlocks of FIG. 12 having different lengths of target-complementary region (arm lengths) on RCA products. Lane 1: 20+20; lane 2: 18+18; lane 3: 16+16; lane 4: 14+14; lane 5: 12+12; lane 6: 10+10; lane 7: 8+8).

### EXAMPLES

Padlock-RCA Materials and Methods

Oligonucleotides

All oligonucleotides were ordered from Integrated DNA Technologies, Inc, and the oligonucleotide sequences are listed in the Table 1. The specific 5′end and 3′end modifications were included in the table if applicable.

1^{st} Padlock Ligation

1^{st} Padlock ligation was performed in a 50 μL reaction by adding 100 nM primer oligonucleotide (SEQ ID NO.1), 100 nM sRCA-p-s3bc padlock oligonucleotide (SEQ ID NO. 10), 1× buffer 4 (New England Biolabs), 0.2 mM ATP, 0.02 U T4 DNA ligase (Thermo-Scientific) and 0.02 mg/ml BSA (New England Biolabs). The reaction was incubated at 37° C. for 30 minutes, followed by heat inactivation at 65° C. for 20 minutes.

1st RCA Products Generation

The first RCA product generation was performed by adding 0.5 μl of the 1^{st} padlock ligation mix to a 50 μl of rolling mix consisting of 1× buffer 4 (New England Biolabs), 1 mM dNTP or 1 mM Uracil containing dNTP sets, 0.1 U phi29 polymerase (Thermo-Scientific), 0.02 mg/ml BSA (New England Biolabs). The reaction was incubated at 37° C. for 75 minutes, followed by heat inactivation at 65° C. for 1 minute.

In Examples 1 to 8, the length of the target-complementary regions in the padlocks used for sRCA is 10+10 nucleotides.

### Example 1

This example investigates ligation and efficiency and the effect of different padlock:RCA monomer ratios.

The general experiment setting is as follows: The first RCA was produced by using a dUTP containing dNTP set. Then different padlock concentrations together with the ligation components were added. After the ligation, the reaction mix was treated with UNG or UNG+Exonuclease I/III to degrade the first RCA products or both the first RCA products and all the unligated linear padlock. Then the samples were analysed with 6% TBE-Urea gel.

Several different amounts of Pre-generated Uracil containing 1^{st} RCA products were added into a 50 μl 2^{nd} padlock ligation mix consisting of 1× buffer 4 (New England Biolabs), 0.2 mM ATP, 0.02 U T4 DNA ligase (Thermo-Scientific) and 0.02 mg/ml BSA (New England Biolabs) and 100 nM p-bx padlock oligonucleotides (SEQ ID No. 11) to achieve a 2^{nd} RCA padlock: 1^{st} RCA monomer of 3:9, 3:1 or 3:0.02. The reaction was incubated at 37° C. for 30 minutes. Then each secondary ligation mix were divided into two portions, one portion was added 0.02 U Uracil-DNA Glycosylase (Thermo-Scientific), 0.2U Endonuclease IV (New England Biolabs), and another portion was added 0.02U Uracil-DNA Glycosylase (Thermo-Scientific), 0.2 U Endonuclease IV (New England Biolabs), 0.4 U Exonuclease I (New England Biolabs), and 4U Exonuclease III (New England Biolabs). The reaction was incubated at 37° C. for 60 minutes. Then 5 μl of each sample was mixed with 5 μl of 2× Novex® TBE-Urea Sample Buffer (Invitrogen), followed by a 80° C. denaturation and placed on ice immediately after the heating. 10 μl of each sample was loaded into each well of 6% TBE-Urea gel (Invitrogen) and run for 60 minutes with 180 Volt. Then the gel was stained with 1× SYBR Gold (Invitrogen) 1× TEB buffer, and imaged with Gel Doc EZ System (Bio-Rad). The results are shown in FIG. 2.

### Example 2

Stability Test

This experiment was performed to test the padlock-sRCA stability at high temperature ex 65° C. and 95° C. 1 μl of 1^{st} RCA products was added into a 50 μl 2^{nd} padlock ligation mix consisting of 1× buffer 4 (New England Biolabs), 0.2 mM ATP, 0.02 U T4 DNA ligase (Thermo-Scientific) and 0.02 mg/ml BSA (New England Biolabs) and 100 nM p-bx padlock oligonucleotides (SEQ ID NO. 11). The reaction was incubated at 37° C. for 30 minutes. Then 1 mM dNTP, 300 nM sRCA-x-primer oligonucleotides and 200 nM sRCA-d-1b-Cy3 oligonucleotides and 0.1 U phi29 polymerase (Thermo-Scientific) were added into the reaction mix followed by 37° C. incubation for 100 minutes. Then the reaction mix were divided into 5 portions and each protion under go with different heating treatment conditions. Then 10 μl of each heating sample was placed on poly-L-lysine slides, and visualized with EPI fluorescence microscopy (Carl Zeiss). The results are shown in FIG. 3, which shows that the padlock sRCA reaction product may be detected after heating at 65° C. and 95° C.

### Example 3

The experiment compares the result of different rounds of RCA using padlock probes, and shows the signal amplification achievable by padlock sRCA; stronger signals are seen with 2 (2RCA) or 3 (3RCA) rounds of RCA.

50 μl of 1 pM 1^{st} padlock ligation mix was incubated with streptavidin coated slides (Surmodics) at 37° C. for 30 minutes, followed by 2 times wash with 50 μl of 1× PBS+0.05% Tween 20. Then 50 μl of the RCA mix consisting of 1× buffer 4 (New England Biolabs), 1 mM dNTP, 0.1 U phi29 polymerase (Thermo-Scientific), 0.02 mg/ml BSA (New England Biolabs) were applied to the slides and incubated at 37° C. for 20 minutes, followed by 2 times wash with 50 μl of 1× PBS+0.05% Tween 20. Thereafter 50 μl of ligation mix consisting of 1× buffer 4 (New England Biolabs), 0.2 mM ATP, 0.02 U T4 DNA ligase (Thermo-Scientific) and 0.02 mg/ml BSA (New England Biolabs) and 100 nM p-bx padlock oligonucleotides (SEQ ID No. 11) were applied on the slides and incubated at 37° C. for 20 minutes, followed by 2 times wash with 50 μl of 1× PBS+0.05% Tween 20. Afterwards 50 μl of the RCA mix consisting of 1× buffer 4 (New England Biolabs), 1 mM dNTP, 0.1 U phi29 polymerase (Thermo-Scientific), 300 nM sRCA-x-primer oligonucleotides (SEQ ID NO. 6) and 0.02 mg/ml BSA (New England Biolabs) were applied to the slides and incubated at 37° C. for 20 minutes, followed by 2 times wash with 50 μl of 1× PBS+0.05% Tween 20. Then the third round ligation mix consisting of 1× buffer 4 (New England Biolabs), 0.2 mM ATP, 0.02 U T4 DNA ligase (Thermo-Scientific) and 0.02 mg/ml BSA (New England Biolabs) and 100 nM p-bw padlock oligonucleotides (SEQ ID NO. 14) were applied on the slides and incubated at 37° C. for 20 minutes, followed by 2 times wash with 50 μl of 1× PBS+0.05% Tween 20. Finally 50 μl of the RCA mix consisting of 1× buffer 4 (New England Biolabs), 1 mM dNTP, 0.1 U phi29 polymerase (Thermo-Scientific), 300 nM sRCA-w-primer oligonucleotides, (SEQ. ID NO. 7) 200 nM sRCA-d-1b-Cy3 oligonucleotides (SEQ ID NO. 3), and 0.02 mg/ml BSA (New England Biolabs) were applied to the slides and incubated at 37° C. for 20 minutes, followed by 2 times wash with 50 μl of 1× PBS+0.05% Tween 20. Depending on the experiment settings, some samples do not have 2 or more rolling steps, then, 200 nM sRCA-d-1b-Cy3 oligonucleotide (SEQ ID NO. 3) was provided in the last rolling step. The results are shown in FIG. 4.

### Example 4

This experiment compares visualisation of padlock sRCA results by EPI and SIM microscopy.

50 μl of 1 pM 1^{st} padlock ligation mix was incubated with streptavidin coated slides (Surmodics) at 37° C. for 30 minutes, followed by 2 times wash with 50 μl of 1× PBS+0.05% Tween 20. Then 50 μl of the RCA mix consisting of 1× buffer 4 (New England Biolabs), 1 mM dNTP, 0.1 U phi29 polymerase (Thermo-Scientific), 0.02 mg/ml BSA (New England Biolabs) were applied to the slides and incubated at 37° C. for 60 minutes, followed by 2 times wash with 50 μl of 1× PBS+0.05% Tween 20. Thereafter 50 μl of ligation mix consisting of 1× buffer 4 (New England Biolabs), 0.2 mM ATP, 0.02 U T4 DNA ligase (Thermo-Scientific) and 0.02 mg/ml BSA (New England Biolabs) and 100 nM p-bx padlock oligonucleotides (SEQ ID NO. 11) were applied on the slides and incubated at 37° C. for 30 minutes, followed by 2 times wash with 50 μl of 1× PBS+0.05% Tween 20. Afterwards 50 μl of the RCA mix consisting of 1× buffer 4 (New England Biolabs) or 1× Olink buffer (Kindly provided by Olink Bioscience), 1 mM dNTP, 0.1 U phi29 polymerase (Thermo-Scientific), 300 nM sRCA-x-primer oligonucleotides (SEQ ID NO. 6), 200 nM sRCA-d-S3-FITC oligonucleotides (SEQ ID NO. 2), 200 nM sRCA-d-1b-Cy3 oligonucleotides (SEQ ID NO. 3) and 0.02 mg/ml BSA (New England Biolabs) were applied to the slides and incubated at 37° C. for 60 minutes, followed by 2 times wash with 50 μl of 1× PBS+0.05% Tween 20. The image was acquired with EPI fluorescence microscopy (Carl Zeiss) or Structured Illumination Microscopy (Carl Zeiss). The results are shown in FIG. 5. The images from SIM and EPI are almost the same; the 1RCA products are not in the round shape, and that causes the stretched morphology in the padlock sRCA samples. Also the RCA buffer could affect the shape of the blobs (compare 2RCA buffer 4 and 2RCA Olink buffer in EPI).

### Example 5

In Situ Proximity Ligation Assay (PLA) using Padlock sRCA.

In this experiment a PLA was proximity ligation assay was performed to detect Lgr5 antigen in situ in as Hs578T breast cancer cell line. The PLA product was detected by padlock sRCA compared to normal RCA (one round).

The experiment almost followed the protocol from Olink Duolink kit (Olink Bioscience). Briefly, 40 μl of 0.3 ng/μl LGR5 N-terminal antibody (Abgent) dilute was applied to the cells, after a 2× 5 min 1× TBS+0.05% Tween 20 wash, then 40 μl of 1:250 dilute of the secondary anti-Rabbit plus and minus probe (Olink Bioscience) mixture was applied on the cells. Thereafter 40 μl of ligation mix consisting of 1× T4 DNA ligase buffer (Thermo-Scientific), 0.2 mM ATP, 0.02 U T4 DNA ligase (Thermo-Scientific) and 0.02 mg/ml BSA (New England Biolabs), 100 nM S3 1852 Backpiece oligonucleotides (SEQ ID NO. 8) and 100 nM S3 splint oligonucleotides (SEQ ID NO. 9) were applied on the slides and incubated at 37° C. for 30 minutes, followed by 2 times wash with 1× TBS+0.05% Tween 20. Afterwards 40 μl of the RCA mix consisting of 1× Olink buffer (Kindly provided by Olink Bioscience), 1 mM dNTP, 0.1 U phi29 polymerase (Thermo-Scientific) and 0.02 mg/ml BSA (New England Biolabs) were applied to the slides and incubated at 37° C. for 60 minutes, followed by 2 times wash with 1× TBS+0.05% Tween 20. Then the secondary ligation mix consisting of 1× T4 DNA ligase buffer (Thermo-Scientific), 0.2 mM ATP, 0.02 U T4 DNA ligase (Thermo-Scientific) and 0.02 mg/ml BSA (New England Biolabs) 100 nM p-S3x padlock oligonucleotides (SEQ ID NO. 12) and 100 nM p-bx padlock oligonucleotides (SEQ ID NO. 11) were applied on the slides and incubated at 37° C. for 30 minutes, followed by 2 times wash with followed by 2 times wash with 1× TBS+0.05% Tween 20. Finally 40 μl of the RCA mix consisting of 1× Olink buffer c 1 mM dNTP, 0.1 U phi29 polymerase (Thermo-Scientific), 300 nM sRCA-w-primer oligonucleotides (SEQ ID NO. 7), 200 nM sRCA-d-1b-Cy3 oligonucleotides SEQ ID NO. 3), 200 nM sRCA-d-53-Cy3 (SEQ ID NO. 4) and 0.02 mg/ml BSA (New England Biolabs) were applied to the slides and incubated at 37° C. for 60 minutes, followed by 2 times wash with 2 times wash with 1× TBS+0.05% Tween 20. Then the images were acquired with EPI fluorescence microscopy (Carl Zeiss). The results are shown in FIG. 6. It can be seen that a stronger signal may be detected in sRCA according to the invention as compared to normal RCA, without an increased background, thereby affording improved sensitivity.

### Example 6

50 μl of 100 fM/50 fM/25 fM/12.5 fM/6.25 fM/3.125 fM 1^{st} padlock ligation mix was incubated with streptavidin coated slides (Surmodics) at 37° C. for 30 minutes, followed by 2 times wash with 50 μl of 1× PBS+0.05% Tween 20. Then 50 μl of the RCA mix consisting of 1× buffer 4 (New England Biolabs), 1 mM dNTP, 0.1 U phi29 polymerase (Thermo-Scientific), 0.02 mg/ml BSA (New England Biolabs) were applied to the slides and incubated at 37° C. for 60 minutes, followed by 2 times wash with 50 μl of 1× PBS+0.05% Tween 20. Thereafter 50 μl of ligation mix consisting of 1× buffer 4 (New England Biolabs), 0.2 mM ATP, 0.02 U T4 DNA ligase (Thermo-Scientific) and 0.02 mg/ml BSA (New England Biolabs) and 100 nM p-bx padlock oligonucleotides (SEQ ID NO. 11) were applied on the slides and incubated at 37° C. for 30 minutes, followed by 2 times wash with 50 μl of 1× PBS+0.05% Tween 20. Afterwards 50 μl of the RCA mix consisting of 1× buffer 4 (New England Biolabs), 1 mM dNTP, 0.1 U phi29 polymerase (Thermo-Scientific), 300 nM sRCA-x-primer oligonucleotides (SEQ ID NO. 6) and 0.02 mg/ml BSA (New England Biolabs) were applied to the slides and incubated at 37° C. for 60 minutes, followed by 2 times wash with 50 μl of 1× PBS+0.05% Tween 20. Thereafter, 50 μl of detection mix consist of 100 nM S3-HRP Detection oligo (SEQ ID NO. 5) in 1× Olink buffer (Kindly provided by Olink Bioscience) was applied on the slide and incubated at 37° C. for 60 minutes followed by 2 times wash with 50 μl of 1× PBS+0.05% Tween 20. Then DAB substrate (Olink Bioscience) was applied on the slides, the mixture was incubated for 15 min and washed away with MQ water. The image was taken by bright field microscopy with either 2.5× objective lens (FIG. 7) or 10× objective lens (FIG. 8). The signals may be visible with a smartphone camera or computer camera with an extra macro lens.

### Example 7

Flow Cytometry Detection

50 μl of first RCA mix consisting of 1 pM/100 fM/10 fM/1 fM 1RCA ligated template together with 1× buffer 4 (New England Biolabs), 1 mM dNTP, 0.1 U phi29 polymerase (Thermo-Scientific) and 0.02 mg/ml BSA (New England Biolabs) was incubated at 37° C. for 75 minutes followed by heat inactivation at 65° C. for 1 minute. Thereafter 0.2 mM ATP, 0.02 U T4 DNA ligase (Thermo-Scientific) and 100 nM p-bx padlock oligonucleotides (SEQ ID NO. 11) were added in the reaction followed by 37° C. incubation for 30 minutes. Then 300 nM sRCA-x-primer oligonucleotides (SEQ ID NO. 6), 200 nM sRCA-d-1b-Cy3 oligonucleotides (SEQ ID NO. 3) and 0.1 U phi29 polymerase (Thermo-Scientific) were added into the reaction mix followed by 37° C. incubation for 100 minutes. Then the reaction mix was diluted by 500 μl C2CA buffer consisting of 50 mM Tris-HCl, 20 mM EDTA, 0.1% Tween 20 and 1M NaCl the sample was analyzed with LSRII flow cytometer (BD Bioscience). The results are shown in FIGS. 9 and 10.

In this experiment, as shown in FIG. 9, the sRCA blobs was labeled only with FITC oligo, so there should be only FITC positive events in the flow cytometry, which was shown in the P2 population. According to previous experiments, all background signals will appear in the area where FITC is smaller than 1000, so only events with a FITC signal value higher than 1000 will be counted as a true signal. When running the samples, the flow cytometry machine was set to “high” flow rate and the sampling time was set to 1 min. In this setting, 1 pM sample got 362,006 events and 100 fM sample got 34,005 events, 10 fM sample got 3,778 events and 1 fM sample got 446 events. For the sample where the first RCA padlock was omitted, there was only 1 event. The dose response in the flow cytometry was quite linear. For the detection efficiency, I pM concentration of the padlock with a total volume of 50 ul, there are 3.01*10⁷ padlock molecules, the machine sampled 60 uL of the 10× diluted reaction mix, so, there are 3.612*10⁶ molecules flowed through the machine, and the number we have detected was around 362,006, so our detection efficiency was around 10%.

In the results shown in FIG. 10, all the experiment procedures were the same and the flow cytometry machine was set to “slow” rate and the sampling time was 1 min. Three samples with different concentration were prepared and each sample was run in the flow cytometry machine three times and the data were recorded. Each event shown with a diamond in FIG. 10 was the average number from three different counts and the CV value was calculated based on the three counts. The CV were all below 5% in all three different concentrations.

### Example 8

RCA Growth Curve

This experiment presents a comparison of a padlock sRCA reaction at 5 pM of first RCA template, as compared with a “normal” RCA (i.e. one round only) at different padlock concentrations.

50 μl of first RCA mix consisting of 5 pM 1RCA ligated template together with 1× buffer 4 (New England Biolabs), 1 mM dNTP, 0.1 U phi29 polymerase (Thermo-Scientific) and 0.02 mg/ml BSA (New England Biolabs) was incubated at 37° C. for 60 minutes followed by heat inactivation at 65° C. for 1 minute. Thereafter 0.2 mM ATP, 0.02 U T4 DNA ligase (Thermo-Scientific) 100 nM p-S3x padlock oligonucleotides (SEQ ID NO. 12) and 100 nM p-bx padlock oligonucleotides (SEQ ID NO. 11) were added in the reaction followed by 37° C. incubation for 30 minutes. Then 300 nM sRCA-x-primer oligo (SEQ ID NO. 6), 1× SYBR Gold (Invitrogen) and 0.1 U phi29 polymerase (Thermo-Scientific) were added. Meanwhile, 100 nM p-bx padlock oligonucleotides (SEQ ID NO. 11) were mixed together with 1× CircLigase II Reaction Buffer (Epicentre), 2.5 mM MnCl₂, 5 U CircLigase II ssDNA Ligase (Epicentre). The reaction mixture was incubated at 60° C. for 60 minutes followed by 80° C. for 60 minutes. 5 nM/1 nM/750 pM/500 pM/250 pM/125 pM/75 pM/50 pM of the self-circulated p-bx oligonucleotides (SEQ ID NO. 11) was mixed with 1× buffer 4 (New England Biolabs), 1 mM dNTP, 0.1 U phi29 polymerase (Thermo-Scientific), 300 nM sRCA-x-primer oligo (SEQ ID NO. 6), 1× SYBR Gold (Invitrogen) and 0.02 mg/ml BSA (New England Biolabs). Thereafter, these samples were analyzed with MX3000 Q-PCR (Stratagene). The Cycling parameters were 120 cycles of 37° C. for 1 minute. The results are shown in FIG. 11.

The straight line curve shows the 5 pM sRCA polymerization speed. The other curves show the normal padlock rolling curve under different padlock concentrations. The slope of each curve stands for the rolling speed. If we assume phi29 polymerase rolling at the same rate in all samples, then the slope would stand for how many padlocks get rolled in each sample. The 5 pM sRCA gives almost the same speed as the SnM normal padlock, so two round sRCA gives 1000 fold increase in the signal.

$$efficiency = \frac{5 nM}{50 pM / 10 \times 1379} \times 100 \% = 72.52 \%$$

### Example 9

This experiment shows how the length of the target-complementary regions (hybridized arm of the padlock on RCA product) affects the rolling efficiency of secondary RCA padlocks.

A series of 90 bp long padlocks was used, having the same backbone and ligation site, but differing in the arm length for the hybridization. The longest arm is 20+20, and for the shorter arms, the border bases were mutated to unmatched bases.

The first RCA products were pre rolled for 1 hr and then have the second RCA padlock ligated on the RCA products for 1 hr. Then the rolling mix and sybr Gold were added into the ligation mix and put into the Q-PCR machine to monitor the growth curve of each reaction. The data was collected every 1 min and lasted for 4 hrs.

50 μl of first RCA mix consisting of 5 pM 1RCA ligated template together with 1× buffer 4 (New England Biolabs), 1 mM dNTP, 0.1 U phi29 polymerase (Thermo-Scientific) and 0.02 mg/ml BSA (New England Biolabs) was incubated at 37° C. for 60 minutes followed by heat inactivation at 65° C. for 1 minute. Thereafter 100 nM of sRCA-p-S3×20 (SEQ ID NO. 15), sRCA-p-S3×18 (SEQ ID NO. 16), sRCA-p-S3×16 (SEQ ID NO. 17), sRCA-p-S3×14 (SEQ ID NO. 18), sRCA-p-S3×12 (SEQ ID NO. 19), sRCA-p-S3×10 (SEQ ID NO. 20), sRCA-p-S3×8 (SEQ ID NO. 21) were separately mixed with 0.2 mM ATP, 0.02 U T4 DNA ligase (Thermo-Scientific) followed by 37° C. incubation for 30 minutes. An aliquot was taken and treated with 0.02 U Uracil-DNA Glycosylase (Thermo-Scientific), 0.2 U Endonuclease IV (New England Biolabs) at 37° C. for 60 minutes, then the reaction mix were analyzed in 10% TBE-Urea gel (Invitrogen). Then the rest of the samples were mixed with 300 nM sRCA-x-primer oligonucleotides, 1× SYBR Gold (Invitrogen) and 0.1 U phi29 polymerase (Thermo-Scientific). Samples were analyzed with MX3000 Q-PCR (Stratagene). The Cycling parameters were 120 cycles of 37° C. for 1 minute.

The results are shown in FIG. 12. It is clear from this that the shorter the length of the arm (target complementary region) the faster the padlock can roll in the second RCA reaction. The 8+8 padlock gave the best results. Comparing the 10+10 padlock with the 12+12 padlock, they have almost the same amount of ligated padlock, but the 10+10 rolls faster. The curve for the 20+20 padlock is almost flat, and does show that the second RCA occurs at a very low, almost unappreciable level. The 18+18 padlock has a slightly higher background, and therefore the signal appears stronger than is actually the case. It is normal practice when evaluating a graph like this to disregard the first few cycles, and to measure the increase from that. Indeed, for a better evaluation of the results, rather than looking at the end point measurements as shown in FIG. 12, a derivative of the growth curves would be prepared, as described in Nilsson et al., 2002, 30(14); e66. Nonetheless, the results show a correlation between length and RCA reaction efficiency. The results show that for a successful and efficient RCA using a padlock probe, the probe should have an arm length (length of target-complementary region) of less than 20 or less than 18. A second RCA may be obtained with an arm length of up to 16, but efficiency may be improved with shorter arm lengths. Accordingly the results support that an arm length of up to or less than 14, or more particularly up to or less than 12 may give better results.

The results of a further experiment carried out to assess ligation efficiency of the different padlocks are shown in FIG. 13. This shows that the length of the arm also has an effect on ligation efficiency and that the shorter padlocks have a slightly better ligation efficiency.

### Example 10

A protocol for carrying out the method of the present invention, wherein a cleaning step (3′ exonucleolysis—step IV) to remove unbound and/or unligated probes (circularisable oligonucleotides) is performed after the ligation of the circularisable probes is provided below.

I—Hybridization and Ligation

| | | final | |
|---|---|---|---|
| | conc | conc | vol |
| Phi29 buffer | 10 x | 1 X | 5 |
| NAD | 10 mM | 0.8 mM | 4 |
| KRAS selector (template conc 10 pM) | 100 nM | 1 nM | 0.5 |
| Ampligase | 5 u/μl | 0.2 u/μl | 2 |
| H₂O | | | 13.5 |
| Vortex and spin down | | total volume | 25.00 |
| 95° C. 5 min | | | |
| 60° C. 2 h | | | |
| Spin down before opening | | | |

II—1RCA

| | | final | |
|---|---|---|---|
| | conc | conc | vol |
| dNTPs (“T”) | 25 mM | 0.25 mM | 0.6 |
| Φ29 polymerase | 10 u/μl | 0.1 u/μl | 0.6 |
| BSA | 10 g/l | 0.4 g/l | 2.4 |
| Phi29 buffer | 10 x | 1 x | 1 |
| H2O | | | 5.40 |
| | | mix volume | 10.00 |
| Vortex tubes and spin down | | total volume | 60.00 |
| 37° C. 90 min | | | |
| 50° C. 10 min | | | |
| 20° C. | | | |
| Spin down before opening | | | |

III—Secondary Padlock Hybridization and Ampligase Ligation

| | | final | |
|---|---|---|---|
| | conc | conc | vol |
| NAD | 100 mM | 0.8 mM | 0.56 |
| 2nd padlock 35G | 10 μM | 0.1 μM | 0.7 |
| 2nd padlock 35A | 10 μM | 0.1 μM | 0.7 |
| Ampligase | 5 u/μl | 0.2 u/μl | 2.8 |
| Phi29 buffer | 10 x | 1 x | 1.00 |
| H2O | | | 4.24 |
| | | mix volume | 10.00 |
| Vortex and spin down | | total volume | 70.00 |
| 45° C. 60 min | | | |
| Spin down before opening | | | |

IV—Secondary Padlock Removal

| | | final | |
|---|---|---|---|
| | conc | conc | vol |
| Φ29 polymerase | 10 u/μl | 0.1 u/μl | 0.80 |
| Phi29 buffer | 10 x | 1 x | 1.00 |
| H2O | | | 8.20 |
| | | mix volume | 10.00 |
| Vortex and spin down | | total volume | 80.00 |
| 37° C. 30 min | | | |
| Spin down before opening | | | |

V—2RCA

| | | final | |
|---|---|---|---|
| | conc | conc | vol |
| Primer | 10 μM | 0.2 μM | 1.80 |
| Detection oligo 35A | 10 μM | 0.1 μM | 0.90 |
| Detection oligo 35G | 10 μM | 0.1 μM | 0.90 |
| dNTPs (“T”) | 25 mM | 0.75 mM | 2.70 |
| Phi29 buffer | 10 x | 1 x | 1.00 |
| H2O | | | 2.70 |
| | | mix volume | 10.00 |
| Vortex and spin down | | total volume | 90.00 |
| 37° C. 90 min | | | |
| Add 2 ul 0.5M EDTA to stop reaction | | | |
| Spin down before opening | | | |

TABLE 1

| SEQ ID | | | | | | |
|---|---|---|---|---|---|---|
| NO. | Name | 5′mod | Sequence | | | 3′mod |
| 1 | primer | bio | AAAAAAAAAA | TATGACAGAAC TAGACACTCTT | | |
| 2 | sRCA-d-S3-FITC | FITC | AAAAAA | CAGTGAATGC GAGTCCGTCT | | UUUU-ome |
| 3 | sRCA-d-1b-Cy3 | Cy3 | AAAAAA | CTAGTGCTGG ATGATCGTCC | | UUUU-ome |
| 4 | sRCA-d-S3-Cy3 | Cy3 | | CAGTGAATGC GAGTCCGTCT | | UUUU-ome |
| 5 | S3-HRP Detection | HRP | | CAGTGAATGC GAGTCCGTCT | | UUUU-ome |
| | oligo | | | | | |
| 6 | sRCA-x-primer | | | GATGTCTGAGGC | | phosphothiol |
| | | | | | | bond for last |
| | | | | | | four bases |
| 7 | sRCA-w-primer | | | CCTCTATTGGTA | | phosphothiol |
| | | | | | | bond for last |
| | | | | | | four bases |
| 8 | S3 1852 Backpiece | Phos | CTATTAGCGTC | CAGTGAATGCGAGTCCGTCTAACTAGT | AAGAGTGTCTA | |
| | | | | GCTGGATGATCGTCC | | |
| 9 | S3 splint | Phos | | GTTCTGTCATATTTAAGCGTCTTAA | | |
| 10 | sRCA-p-s3bc | Phos | GTTCTGTCATA | CAGTGAATGCGAGTCCGTCT AA | AAGAGTGTCTA | |
| | | | | CTAGTGCTGGATGATCGTCC AA | | |
| | | | | AGCGATCTGCGAGACCGTAT | | |
| 11 | sRCA-p-1bx | Phos | ATGATCGTCCTT | ATCACTGCCCCG ACAGGCCTCAGA | TCCTAGTGCTGG | |
| | | | | CATCATAATAGC | | |
| 12 | sRCA-p-1S3x | Phos | GAGTCCGTCTTT | ATCACTGCCCCG ACAGGCCTCAGA | TCCAGTGAATGC | |
| | | | | CATCATAATAGC | | |
| 13 | sRCA-p-1S3w | Phos | GAGTCCGTCTTT | ATCCTCAATGCT GCTGCTGTAC | TCCAGTGAATGC | |
| | | | | TACCAATAGA GGTA | | |
| 14 | sRCA-p-1bw | Phos | ATGATCGTCCTT | CCTCAATGCT GCTGCTGTAC | TCCTAGTGCTGG | |
| | | | | TACCAATAGA GGTA | | |
| 15 | sRCA-p-S3x20 | Phos | GAGTCCGTCTAA | ATCACTGCCCCG ACAGGCCTCAGA | TTCTGTCATACA | |
| | | | CTAGTGCT | CATCATAATAGC CTCTCTCTCTCTCT | GTGAATGC | |
| 16 | sRCA-p-S3x18 | Phos | GAGTCCGTCTAA | ATCACTGCCCCG ACAGGCCTCAGA | CACTGTCATACA | |
| | | | CTAGTGTC | CATCATAATAGC CTCTCTCTCTCTCT | GTGAATGC | |
| 17 | sRCA-p-S3x16 | Phos | GAGTCCGTCTAA | ATCACTGCCCCG ACAGGCCTCAGA | CATCGTCATACA | |
| | | | CTAGCTTC | CATCATAATAGC CTCTCTCTCTCTCT | GTGAATGC | |
| 18 | sRCA-p-S3x14 | Phos | GAGTCCGTCTAA | ATCACTGCCCCG ACAGGCCTCAGA | CATCTCCATACA | |
| | | | CTCTCTTC | CATCATAATAGC CTCTCTCTCTCTCT | GTGAATGC | |
| 19 | sRCA-p-S3x12 | Phos | GAGTCCGTCTAA | ATCACTGCCCCG ACAGGCCTCAGA | CATCTCTCTACA | |
| | | | TCCTCTTC | CATCATAATAGC CTCTCTCTCTCTCT | GTGAATGC | |
| 20 | sRCA-p-S3x10 | Phos | GAGTCCGTCTTC | ATCACTGCCCCG ACAGGCCTCAGA | CATCTCTCCTCA | |
| | | | TCCTCTTC | CATCATAATAGC CTCTCTCTCTCTCT | GTGAATGC | |
| 21 | sRCA-p-S3x8 | Phos | GAGTCCGTTCTC | ATCACTGCCCCG ACAGGCCTCAGA | CATCTCTCCTTC | |
| | | | TCCTCTTC | CATCATAATAGC CTCTCTCTCTCTCT | GTGAATGC | |

1. A method for performing a localised RCA reaction comprising at least two rounds of RCA, wherein the product of a second RCA reaction is attached, and hence localised, to a product of a first RCA reaction, said method comprising:
(a) providing a concatemeric first RCA product comprising repeated monomers;
(b) directly or indirectly hybridising to monomers of said first RCA product a circularisable oligonucleotide comprising target-complementary 3′ and 5′ end regions such that the 3′ and 5′ ends of said oligonucleotide hybridise in juxtaposition for ligation directly or indirectly to each other, wherein the target is a sequence in a monomer of said first RCA product or an intermediate molecule hybridised thereto, and wherein the target-complementary end regions of said circularisable oligonucleotide are 6 to 16 nucleotides in length;
(c) directly or indirectly ligating the ends of said circularisable oligonucleotide to circularise the oligonucleotide, thereby to provide a template for a second RCA reaction, wherein when said ends are indirectly ligated (i) either a gap oligonucleotide is provided which hybridises to the monomers of the first RCA product in between the 3′ and 5′ ends of the circularisable oligonucleotide such that it may be ligated to the respective ends, or the hybridised 3′ end of the circularisable oligonucleotide is extended by a polymerase such that the extended 3′ end may be ligated to the hybridised 5′ end, and (ii) the total length of the region of the second RCA template directly or indirectly hybridised to the monomers is no longer than 32 nucleotides in length; and
(d) performing a second RCA reaction using said second RCA template of (c) and a primer for said second RCA, to form a second RCA product, wherein in said second RCA reaction the second RCA template remains attached to the first RCA product, and thereby the second RCA product is attached to the first RCA product.

2. The method of claim 1, wherein the circularisable oligonucleotide hybridises directly to the first RCA product.

3. The method of claim 1, wherein step (a) comprises generating a first RCA product.

4. The method of claim 1, wherein steps (a), (b), (c) and (d) are repeated one or more times.

5. The method of claim 1, wherein the target-complementary regions of the circularisable oligonucleotide are 6-15, 6-14, 6-13, 6-12, 6-11, or 6-10 nucleotides in length.

6. The method of any claim 1, wherein the target-complementary regions of the circularisable oligonucleotide are 7-12, 7-11, 7-10 or 7-9 nucleotides in length.

7. The method of claim 1, wherein the total length of the hybridised region of the second RCA template is no longer than 30, 28, 26, 24, 20, 19, 18, 17, or 16 nucleotides.

8. The method of claim 1, wherein the primer for the second RCA reaction is provided after hybridisation or after ligation of the circularisable oligonucleotide.

9. The method of claim 1, wherein the method is homogenous.

10. The method of claim 1, wherein the method is performed in a solid-phase based format.

11. The method of claim 10, wherein the first RCA product is immobilised.

12. The method of claim 1, wherein said method comprises one or more washing steps after hybridisation and/or ligation of the circularisable oligonucleotide.

13. The method of claim 12, wherein a step of stringent washing is performed after step (c) but before step (d) to remove any unligated oligonucleotides.

14. The method of claim 1, wherein blocking oligonucleotides are hybridised to the first RCA product in between the circularisable oligonucleotides.

15. The method of claim 1, wherein a component having 3′ exonuclease activity is provided after the ligation of the circularisable oligonucleotide.

16. The method of claim 15 wherein the component having 3′ exonuclease activity is provided prior to the addition of the primer in step (d).

17. The method of claim 1, further comprising detecting a said attached second RCA product.

18. A method for detecting an analyte in a sample, wherein a first circular RCA template is generated from a nucleic acid analyte, or is used or generated as a marker for a said analyte, said method comprising performing a first RCA reaction using said first RCA template to generate a first RCA product, performing a localised second RCA reaction as defined in any one of claims 1 to 17, and detecting said second RCA product, and optionally said first RCA product, thereby to detect said analyte.

19. The method of claim 18, wherein the first RCA product is generated by an immunoRCA or proximity probe assay.

20. The method of claim 18 for detecting more than one analyte in a sample, wherein different circularisable oligonucleotides are provided for each analyte and each comprises a different reporter domain for detection of the second RCA product, or said analytes are detected sequentially.

21. The method of claim 18, wherein the RCA product is detected using a labelled detection oligonucleotide which hybridises specifically to the RCA product, by using a nucleic acid stain or by using labelled nucleotides for incorporation into the RCA product.

22. The method of claim 18, wherein the RCA product is detected using liquid chromatography, electrophoresis, mass spectrometry, microscopy, real-time PCR, fluorescent probes, microarray, colorimetric analysis such as ELISA, flow cytometry, mass spectrometry (CyTOF) or by turbidometric, magnetic, particle counting, electric, surface sensing, and weight-based detection techniques.


SEQUENCE LISTING (21 sequences)

SEQ ID NO: 1 - DNA, 32 bp, Artificial Sequence
  Primer
aaaaaaaaaatatgacagaactagacactctt

SEQ ID NO: 2 - DNA, 30 bp, Artificial Sequence
  sRCA-d-S3-FITC
aaaaaacagtgaatgcgagtccgtctuuuu

SEQ ID NO: 3 - DNA, 30 bp, Artificial Sequence
  sRCA-d-1b-Cy3
aaaaaactagtgctggatgatcgtccuuuu

SEQ ID NO: 4 - DNA, 24 bp, Artificial Sequence
  sRCA-d-S3-Cy3
cagtgaatgcgagtccgtctuuuu

SEQ ID NO: 5 - DNA, 24 bp, Artificial Sequence
  S3-HRP Detection oligo
cagtgaatgcgagtccgtctuuuu

SEQ ID NO: 6 - DNA, 12 bp, Artificial Sequence
  sRCA-x-primer
gatgtctgaggc

SEQ ID NO: 7 - DNA, 12 bp, Artificial Sequence
  sRCA-w-primer
cctctattggta

SEQ ID NO: 8 - DNA, 64 bp, Artificial Sequence
  S3 1852 Backpiece
ctattagcgtccagtgaatgcgagtccgtctaactagtgctggatgatcgtccaagagtgtcta

SEQ ID NO: 9 - DNA, 25 bp, Artificial Sequence
  S3 splint
gttctgtcatatttaagcgtcttaa

SEQ ID NO: 10 - DNA, 86 bp, Artificial Sequence
  sRCA-p-s3bc
gttctgtcatacagtgaatgcgagtccgtctaactagtgctggatgatcgtccaaagcgatctgcgagaccgtataagagtgtcta

SEQ ID NO: 11 - DNA, 60 bp, Artificial Sequence
  sRCA-p-1bx
atgatcgtccttatcactgccccgacaggcctcagacatcataatagctcctagtgctgg

SEQ ID NO: 12 - DNA, 60 bp, Artificial Sequence
  sRCA-p-1S3x
gagtccgtctttatcactgccccgacaggcctcagacatcataatagctccagtgaatgc

SEQ ID NO: 13 - DNA, 60 bp, Artificial Sequence
  sRCA-p-1S3w
gagtccgtctttatcctcaatgctgctgctgtactaccaatagaggtatccagtgaatgc

SEQ ID NO: 14 - DNA, 58 bp, Artificial Sequence
  sRCA-p-1bw
atgatcgtccttcctcaatgctgctgctgtactaccaatagaggtatcctagtgctgg

SEQ ID NO: 15 - DNA, 90 bp, Artificial Sequence
  sRCA-p-S3x20
gagtccgtctaactagtgctatcactgccccgacaggcctcagacatcataatagcctctctctctctctttctgtcatacagtgaatgc

SEQ ID NO: 16 - DNA, 90 bp, Artificial Sequence
  sRCA-p-S3x18
gagtccgtctaactagtgtcatcactgccccgacaggcctcagacatcataatagcctctctctctctctcactgtcatacagtgaatgc

SEQ ID NO: 17 - DNA, 90 bp, Artificial Sequence
  sRCA-p-S3x16
gagtccgtctaactagcttcatcactgccccgacaggcctcagacatcataatagcctctctctctctctcatcgtcatacagtgaatgc

SEQ ID NO: 18 - DNA, 90 bp, Artificial Sequence
  sRCA-p-S3x14
gagtccgtctaactctcttcatcactgccccgacaggcctcagacatcataatagcctctctctctctctcatctccatacagtgaatgc

SEQ ID NO: 19 - DNA, 90 bp, Artificial Sequence
  sRCA-p-S3x12
gagtccgtctaatcctcttcatcactgccccgacaggcctcagacatcataatagcctctctctctctctcatctctctacagtgaatgc

SEQ ID NO: 20 - DNA, 90 bp, Artificial Sequence
  sRCA-p-S3x10
gagtccgtcttctcctcttcatcactgccccgacaggcctcagacatcataatagcctctctctctctctcatctctcctcagtgaatgc

SEQ ID NO: 21 - DNA, 90 bp, Artificial Sequence
  sRCA-p-S3x8
gagtccgttctctcctcttcatcactgccccgacaggcctcagacatcataatagcctctctctctctctcatctctccttcgtgaatgc